(19) United States
(12) Patent Application Publication
(10) Pub. No.: US 2020/0316092 A1
(43) Pub. Date: Oct. 8, 2020

(54) METHODS OF DIAGNOSIS AND TREATMENT OF LIVER DISEASES USING OBETICHOLIC ACID

(71) Applicant: Intercept Pharmaceuticals, Inc., New York, NY (US)

(72) Inventors: Jeffrey EDWARDS, Del Mar, CA (US); Greg EVERSON, Greenwood Village, CO (US)

(21) Appl. No.: 16/831,365

(22) Filed: Mar. 26, 2020

Related U.S. Application Data
(60) Provisional application No. 62/824,254, filed on Mar. 26, 2019.

(51) Int. Cl. A61K 31/575; G01N 33/94

(52) U.S. Cl. CPC A61K 31/575; G01N 2800/085; G01N 2800/52; G01N 33/94

(57) ABSTRACT

The disclosure relates to methods of using obeticholic acid for the treatment of various diseases and conditions.

### RELATED APPLICATIONS

This application claims priority to and the benefit of U.S. Provisional Application No. 62/824,254, filed on Mar. 26, 2019, the entire contents of which are incorporated herein by reference.

### BACKGROUND

Nonalcoholic fatty liver disease (NAFLD) is considered to be a hepatic manifestation of metabolic syndrome, a cluster of closely related clinical features linked to visceral obesity and characterized by insulin resistance, dyslipidemia, and hypertension. NAFLD is the most common cause of chronic liver disease in the western hemisphere, and its prevalence is expected to rise. NAFLD is thought to be represented by a spectrum of histologically-defined diseases, which progresses from simple steatosis to nonalcoholic steatohepatitis (NASH). NASH is characterized by hepatocellular injury, inflammation, and progressive fibrosis potentially leading to cirrhosis, hepatic decompensation, hepatocellular carcinoma (HCC), and liver-related death. Of all the histologic features of NASH, fibrosis is considered the strongest predictor of adverse clinical outcomes.

There are currently no approved therapies for the treatment of NASH or compensated cirrhosis due to NASH nor an accepted standard of care. With no approved treatments targeting the underlying pathophysiology, the current treatment strategy for NASH patients with cirrhosis becomes largely supportive. It is therefore critical to develop effective therapies that can reverse fibrosis or prevent the progression of fibrosis to cirrhosis, with the goal of reducing complications secondary to cirrhosis ultimately improving quality of life and liver-transplant-free survival.

Obeticholic acid (OCA) is a modified bile acid and farnesoid X receptor (FXR) agonist. OCA's potent FXR agonist effects make it an attractive therapeutic agent for NASH due to its multiple FXR-mediated effects including an increase in insulin sensitivity, glucose and lipid metabolism, hepatocyte protection against bile-acid-induced cytotoxicity, anti-inflammatory effects in liver and vasculature, and prevention and reversal of liver fibrosis. Nonclinical studies have shown several potentially beneficial properties of FXR agonism in NASH with fibrosis and compensated cirrhosis due to NASH.

### SUMMARY

The present disclosure relates to methods of using obeticholic acid for treating a disease or condition. In certain instances, the disease or condition is chronic liver disease, nonalcoholic fatty liver disease (NAFLD), nonalcoholic steatohepatitis (NASH), hepatitis C infection, alcoholic liver disease, liver damage due to progressive fibrosis, or liver fibrosis. In another instance, the disease is NASH. In still other instances, the disease or condition is solid-tumor cancer such as, for example, liver cancer (e.g., hepatocellular carcinoma (HCC) or cholangiocarcinoma). Further provided herein are novel dosing regimens for administration of obeticholic acid for treatment of the diseases or conditions described herein.

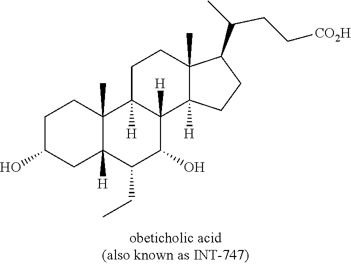

A first aspect of the disclosure relates to a method of treating a disease or condition described herein in a subject in need thereof, comprising administering to the subject a therapeutically effective amount of obeticholic acid, or a pharmaceutically acceptable salt, ester, or amino acid conjugate thereof.

Another aspect of the disclosure relates to a method of treating NAFLD in a subject in need thereof, comprising administering to the subject a therapeutically effective amount of obeticholic acid, or a pharmaceutically acceptable salt, ester, or amino acid conjugate thereof.

Another aspect of the disclosure relates to a method of treating NASH in a subject in need thereof, comprising administering to the subject a therapeutically effective amount of obeticholic acid, or a pharmaceutically acceptable salt, ester, or amino acid conjugate thereof.

Another aspect of the disclosure relates to a method of slowing down or reversing the progression of NASH in a subject in need thereof, comprising administering to the subject a therapeutically effective amount of obeticholic acid, or a pharmaceutically acceptable salt, ester, or amino acid conjugate thereof.

Another aspect of the disclosure relates to a method of slowing down or reversing the progression of liver fibrosis in a subject in need thereof, comprising administering to the subject a therapeutically effective amount of obeticholic acid, or a pharmaceutically acceptable salt, ester, or amino acid conjugate thereof.

Another aspect of the disclosure relates to a method of slowing down or reversing the progression of cirrhosis (e.g., compensated cirrhosis) in a subject in need thereof, comprising administering to the subject a therapeutically effective amount of obeticholic acid, or a pharmaceutically acceptable salt, ester, or amino acid conjugate thereof.

Another aspect of the disclosure relates to use of obeticholic acid, or a pharmaceutically acceptable salt, ester, or amino acid conjugate thereof, for treating NAFLD, treating NASH, slowing down or reversing the progression of NASH, slowing down or reversing the progression of liver fibrosis, and/or slowing down or reversing the progression of cirrhosis (e.g., compensated cirrhosis).

Another aspect of the disclosure relates to obeticholic acid, or a pharmaceutically acceptable salt, ester, or amino acid conjugate thereof for use in treating NAFLD, treating NASH, slowing down or reversing the progression of NASH, slowing down or reversing the progression of liver fibrosis, and/or slowing down or reversing the progression of cirrhosis (e.g., compensated cirrhosis).

Another aspect of the disclosure relates to use of obeticholic acid, or a pharmaceutically acceptable salt, ester, or amino acid conjugate thereof, in the manufacture of a medicament for treating NAFLD, treating NASH, slowing down or reversing the progression of NASH, slowing down or reversing the progression of liver fibrosis, and/or slowing down or reversing the progression of cirrhosis (e.g., compensated cirrhosis).

Another aspect of the disclosure relates to obeticholic acid, or a pharmaceutically acceptable salt, ester, or amino acid conjugate thereof for use in the manufacture of a medicament for treating NAFLD, treating NASH, slowing down or reversing the progression of NASH, slowing down or reversing the progression of liver fibrosis (e.g., NASH with fibrosis), and/or slowing down or reversing the progression of cirrhosis (e.g., compensated cirrhosis due to NASH).

Another aspect of the disclosure relates to a method of treating a disease in a subject, wherein the method comprises: a) determining a Disease Severity Index (DSI) score using a HepQuant® test (e.g, HepQuant®-SHUNT test); b) comparing the DSI score to a predetermined cutoff value; and c) administering to the subject at least one therapeutically effective amount of obeticholic acid, or a pharmaceutically acceptable salt, ester, or amino acid conjugate thereof when the DSI score is equal to or greater than the predetermined cutoff value.

Another aspect of the disclosure relates to a method of treating a disease in a subject, wherein the method comprises: a) determining a first DSI score using a HepQuant® test (e.g, HepQuant®-SHUNT test) at a time point prior to the administration of at least one therapeutically effective amount of obeticholic acid, or a pharmaceutically acceptable salt, ester, or amino acid conjugate thereof; b) administering to the subject at least one therapeutically effective amount of obeticholic acid, or a pharmaceutically acceptable salt, ester, or amino acid conjugate thereof; c) determining at least a second DSI score using a HepQuant® test (e.g, HepQuant®-SHUNT test) at at least one time point after the administration of the at least one therapeutically effective amount of obeticholic acid, or a pharmaceutically acceptable salt, ester, or amino acid conjugate thereof; d) determining a DSI difference score by calculating the difference between the at least second DSI score and the first DSI score; e) comparing the DSI difference score to a predetermined cutoff value; 0 discontinuing administration of the at least one therapeutically effective amount of obeticholic acid when the DSI difference score is less than or equal to the predetermined cutoff value.

Another aspect of the disclosure relates to a method of treating a disease in a subject, wherein the method comprises: a) determining a first DSI score using a HepQuant® test (e.g, HepQuant®-SHUNT test) at a time point prior to the administration of at least one therapeutically effective amount of obeticholic acid, or a pharmaceutically acceptable salt, ester, or amino acid conjugate thereof; b) administering to the subject at least one therapeutically effective amount of obeticholic acid, or a pharmaceutically acceptable salt, ester, or amino acid conjugate thereof; c) determining at least a second DSI score using a HepQuant® test (e.g, HepQuant®-SHUNT test) at at least one time point after the administration of the at least one therapeutically effective amount of obeticholic acid, or a pharmaceutically acceptable salt, ester, or amino acid conjugate thereof; d) determining a DSI difference score by calculating the difference between the at least second DSI score and the first DSI score; e) comparing the DSI difference score to a predetermined cutoff value; 0 continuing administration of the at least one therapeutically effective amount of obeticholic acid when the DSI difference score is greater than or equal to the predetermined cutoff value.

Another aspect of the disclosure relates to a method of treating a disease in a subject, wherein the method comprises: a) determining a first DSI score using a HepQuant® test (e.g, HepQuant®-SHUNT test) at a time point prior to the administration of at least one first therapeutically effective amount of obeticholic acid, or a pharmaceutically acceptable salt, ester, or amino acid conjugate thereof; b) administering to the subject at least one first therapeutically effective amount of obeticholic acid, or a pharmaceutically acceptable salt, ester, or amino acid conjugate thereof; c) determining at least a second DSI score using a HepQuant® test (e.g, HepQuant®-SHUNT test) at at least one time point after the administration of the at least one first therapeutically effective amount of obeticholic acid, or a pharmaceutically acceptable salt, ester, or amino acid conjugate thereof; d) determining a DSI difference score by calculating the difference between the at least second DSI score and the first DSI score; e) comparing the DSI difference score to a predetermined cutoff value; and f) administering to the subject at least one second therapeutically effective amount of obeticholic acid, or a pharmaceutically acceptable salt, ester, or amino acid conjugate thereof when the DSI difference score is greater than or equal to the predetermined cutoff value.

Another aspect of the disclosure relates to a method of treating a disease in a subject, the method comprising: a) determining a first DSI score using a HepQuant® test (e.g, HepQuant®-SHUNT test) at a time point prior to the administration of at least one first therapeutically effective amount of obeticholic acid, or a pharmaceutically acceptable salt, ester, or amino acid conjugate thereof; b) administering to the subject at least one first therapeutically effective amount of obeticholic acid, or a pharmaceutically acceptable salt, ester, or amino acid conjugate thereof; c) determining at least a second DSI score using a HepQuant® test (e.g, HepQuant®-SHUNT test) at at least one time point after the administration of the at least one first therapeutically effective amount of obeticholic acid, or a pharmaceutically acceptable salt, ester, or amino acid conjugate thereof; d) determining a DSI difference score by calculating the difference between the at least second DSI score and the first DSI score; e) comparing the DSI difference score to a predetermined cutoff value; f) administering to the subject at least one second therapeutically effective amount of obeticholic acid, or a pharmaceutically acceptable salt, ester, or amino acid conjugate thereof when the DSI difference score is less than or equal to the predetermined cutoff value.

The methods of the present application address unmet needs in the treatment or prevention of a disease or condition, such as those described herein.

### BRIEF DESCRIPTION OF THE DRAWINGS

FIG. 1 is a cartoon schematic of liver function as measured by a HepQuant®-SHUNT test.

FIG. 2 is a graph of systemic HFR versus Portal HFR and the corresponding DSI values.

FIG. 3 is a chart showing the correlation between DSI scores and the probability of varices in subjects with NASH or HCV infection.

FIG. 4 is an outline of a phase 1, double-blind, randomized, placebo-controlled study to evaluate safety, pharmacokinetics, and pharmacodynamics of obeticholic acid in subjects with nonalcoholic steatohepatitis with fibrosis.

FIG. 5 is a series of graphs showing the total OCA (ng/ml) in samples from healthy and NASH subjects administered OCA at a dose of 10 mg QD or 25 mg QD on day 1 and on day 85.

FIG. 6 is a series of graphs showing the total OCA (ng/ml) in samples from NASH subjects with fibrosis scores of either F1, F2/F3 or F4 administered OCA at a dose of 10 mg QD or 25 mg QD on day 1 of the present study and on day 85.

FIG. 7 is a graph showing the percent change from baseline (Day 85 compared to baseline) in C4 levels, bile acid levels and FGF-19 levels in the three treatment groups.

FIG. 8 is a graph showing the percent change from baseline (Day 85 compared to baseline) in ALT, AST, GGT, total bilirubin, direct bilirubin and ALP levels in the three treatment groups.

FIG. 9 is a series of graphs showing the liver exposure of OCA and its conjugates in liver and plasma samples from NASH subjects with fibrosis scores of F1 or F2/F3 that were administered 10 mg OCA QD.

FIG. 10 is a series of graphs showing the liver exposure of OCA and its conjugates in liver and plasma samples from NASH subjects with fibrosis scores of F1, F2/F3 or F4 that were administered 25 mg OCA QD.

FIG. 11 is a graph showing the liver exposure of OCA and its conjugates in liver samples of NASH subjects with fibrosis scores of F1, F2/F3 or F4 after normalization by dose

FIG. 12 is a schematic showing the timing of HepQuant® assessments performed in the phase 1 trial.

FIG. 13 is a schematic showing the number of subjects in the phase 1 trial that were administered a HepQuant® test on Day −1 (baseline), Day 8 and Day 85 of the phase 1 trial.

FIG. 14 is a graph showing baseline DSI score in NASH subjects having a fibrosis score of F2/F3 in the three treatment groups.

FIG. 15 is a graph showing the Baseline DSI score in NASH subjects having a fibrosis scores of F1, F2/F3 or F4 in the three treatment groups.

FIG. 16 is a graph showing the change in DSI score (Day 85−Baseline [Day 1]) in NASH subjects having a fibrosis score of F2/F3 in the three treatment group.

FIG. 17 is a graph showing the change in DSI score (ΔDSI, Day 85−Baseline [Day 1]) in NASH subjects having a fibrosis score of F1, F2/F3 or F4 in the three treatment groups.

### DETAILED DESCRIPTION

The present application is directed to methods of using obeticholic acid, a pharmaceutically active ingredient (also known as INT-747) having the chemical structure:

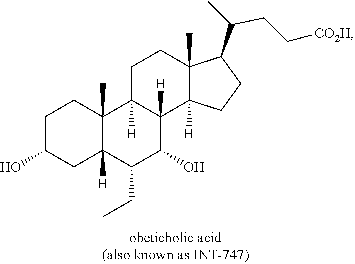

or a pharmaceutically acceptable salt, ester, or amino acid conjugate (such as, e.g., glycine, taurine or sarcosine conjugate) thereof, in the treatment of a disease or condition, such as an FXR mediated disease or disorder.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present specification will control. All patents and publications cited in this specification are incorporated herein by reference in their entireties.

All percentages and ratios used herein, unless otherwise indicated, are by weight.

The term “organ” refers to a differentiated structure (as in a heart, lung, kidney, liver, etc.) consisting of cells and tissues and performing some specific function in an organism. This term also encompasses bodily parts performing a function or cooperating in an activity (e.g., an eye and related structures that make up the visual organs). The term “organ” further encompasses any partial structure of differentiated cells and tissues that is potentially capable of developing into a complete structure (e.g., a lobe or a section of a liver).

As used herein the term “6-ethyl chenodeoxycholic acid”, “6-ECDCA”, “obeticholic acid” or “OCA” refers to a compound having the chemical structure:

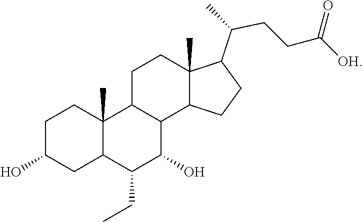 [SMILES: CC[C@@H]1C2C[C@H](O)CC[C@]2(C)C2CC[C@@]3(C)C(CCC3[C@H](C)CCC(=O)O)C2[C@@H]1O]

Other chemical names for obeticholic acid include: 3α,7α-dihydroxy-6α-ethyl-5β-cholan-24-oic acid, 6α-ethyl-chenodeoxycholic acid, 6-ethyl-CDCA, 6ECDCA, cholan-24-oic acid, 6-ethyl-3,7-dihydroxy-,(3α,5β, 6α,7α)- and INT-747. The CAS registry number for obeticholic acid is 459789-99-2. This term refers to all forms of obeticholic acid, e.g., non-crystalline, crystalline and substantially pure.

An “obeticholic acid composition” described herein refers to obeticholic acid administered to a patient in any form described herein including as a component of a pharmaceutical composition.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

“Treating”, includes any effect, e.g., lessening, reducing, modulating, or eliminating, that results in the improvement of the condition, disease, disorder, etc. “Treating” or “treatment” of a disease state includes: arresting the development of the disease state or its clinical symptoms; or relieving the disease state, i.e., causing temporary or permanent regression of the disease state or its clinical symptoms.

The term “regimen” refers to a protocol for dosing and/or timing the administration of one or more therapies (e.g., an obeticholic acid composition described herein for treating a disease, disorder, or condition described herein. A regimen can include periods of active administration and periods of rest as known in the art. Active administration periods include administration of the obeticholic acid compositions described herein in a defined course of time, including, for example, the number of and timing of dosages of the compositions. In some regimens, one or more rest periods can be included where no compound is actively administered, and in certain instances, includes time periods where the efficacy of such compounds can be minimal.

“Preventing” the disease state includes causing the clinical symptoms of the disease state not to develop in a subject that may be exposed to or predisposed to the disease state, but does not yet experience or display symptoms of the disease state.

The term “inhibiting” or “inhibition,” as used herein, refers to any detectable positive effect on the development or progression of a disease or condition. Such a positive effect may include the delay or prevention of the onset of at least one symptom or sign of the disease or condition, alleviation or reversal of the symptom(s) or sign(s), and slowing or prevention of the further worsening of the symptom(s) or sign(s).

“Disease state” means any disease, disorder, condition, symptom, or indication.

The term “effective amount” as used herein refers to an amount of obeticholic acid (e.g., an FXR-activating ligand) that produces an acute or chronic therapeutic effect upon appropriate dose administration. The effect includes the prevention, correction, inhibition, or reversal of the symptoms, signs and underlying pathology of a disease/condition (e.g., fibrosis of the liver, kidney, or intestine) and related complications to any detectable extent.

“A therapeutically effective amount” means the amount of obeticholic acid that, when administered to a mammal for treating a disease, is sufficient to effect such treatment for the disease. The “therapeutically effective amount” will vary depending on obeticholic acid, the disease and its severity and the age, weight, etc., of the mammal to be treated. A therapeutically effective amount can refer to a starting dose or adjusted dose as set forth herein.

A therapeutically effective amount of obeticholic acid can be formulated with a pharmaceutically acceptable carrier for administration to a human or an animal. Accordingly, obeticholic acid or its formulations can be administered, for example, via oral, parenteral, or topical routes, to provide an effective amount of the compound. In alternative embodiments, obeticholic acid prepared in accordance with the present disclosure can be used to coat or impregnate a medical device, e.g., a stent.

For any compound, the therapeutically effective amount can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

A “starting dose” as used herein refers to an initial dose provided to a patient to provide a clinical effect while minimizing onset or occurrence of an adverse effect. A starting dose can, in certain instances, be less than an amount typically administered to a patient. A starting dose is provided in an amount that is titrated or gradually increased over the course of a titration period or during the course of treatment with an obeticholic acid composition described herein.

A “titration period” refers to a length of time for which a starting dose is administered to a patient. A titration period continues for a specified length of time, where the patient is often monitored for liver function and/or liver biochemistry as described herein. In one embodiment a titration period concludes when a patient tolerates an obeticholic acid composition described herein but has a decreased or minimal reduction in alkaline phosphatase.

An “adjusted dose” as used herein refers to a dose of an obeticholic acid composition described herein administered after the termination of a titration period. An adjusted dose is often increased compared to a starting dose but, as provided herein, patient tolerance and other factors described herein determine the dosage amount of an adjusted dose. A “re-adjusted dose” as used herein refers to any changed dosage amount or dose frequency of an adjusted dose in a patient.

“Up titration” refers to increasing the dosage after a starting dose, for example to achieve certain therapeutic effects. The amount increased is determined according to patient tolerance and other factors described herein. The dosage increase may be carried out by increasing the per dose amount and/or dosing frequency.

“Down titration” refers to decreasing the dosage after a starting dose, for example to avoid or reduce certain undesirable side effects. The amount decreased is determined according to patient tolerance and other factors described herein. The dosage decrease may be carried out by decreasing the per dose amount and/or dosing frequency.

“Hepatic impairment” is used in accordance with its standard meaning(s) in the art and can, in certain embodiments herein refer to scoring based upon the Child-Pugh Score of A, B, and C.

Child-Pugh (CP) classification is widely used as a prognostic indicator of hepatic impairment and cirrhosis in addition to distinguishing the disease by clinical phases. CP utilizes 2 clinical parameters (hepatic encephalopathy and ascites) and three laboratory values (bilirubin, albumin, and prothrombin time [PT]/international randomized ratio [INR]). Patients are classified as Class A (mild), Class B (moderate), or Class C (severe) based on their total CP score.

Liver biochemistry (including ALP, ALT, AST, GGT, total and conjugated bilirubin, creatinine, and albumin), prothrombin time (PT)/INR, serum electrolytes, and assessment of Child Pugh (only if the subject is at the study site) and MELD scores can be also monitored to evaluate the safety of the treatment. Model for End-Stage Liver Disease (MELD) is a scoring system used to assess the severity of chronic liver disease.

Various biomarkers can be measured to determine the presence and severity of liver diseases. These biomarkers include bilirubin, albumin, and prothrombin. Bilirubin is made during normal breakdown of red blood cells. Bilirubin passes through the liver and is excreted out of the body. Bilirubin level can be measured through a blood test. Higher than normal levels of bilirubin may indicate liver problems. Albumin is a protein made by the liver. An albumin test may be ordered as part of a liver panel to evaluate liver function. The typical value for serum albumin in blood is 3.4 to 5.4 grams per deciliter. Low albumin levels can indicate a number of health conditions, including liver diseases. Prothrombin time (PT) measures how long it takes blood to form a clot, and is a universal indicator of liver disease severity. In addition, the serum level of cortisol or fibrinogen a chain may be assessed to determine liver function and the presence of liver diseases.

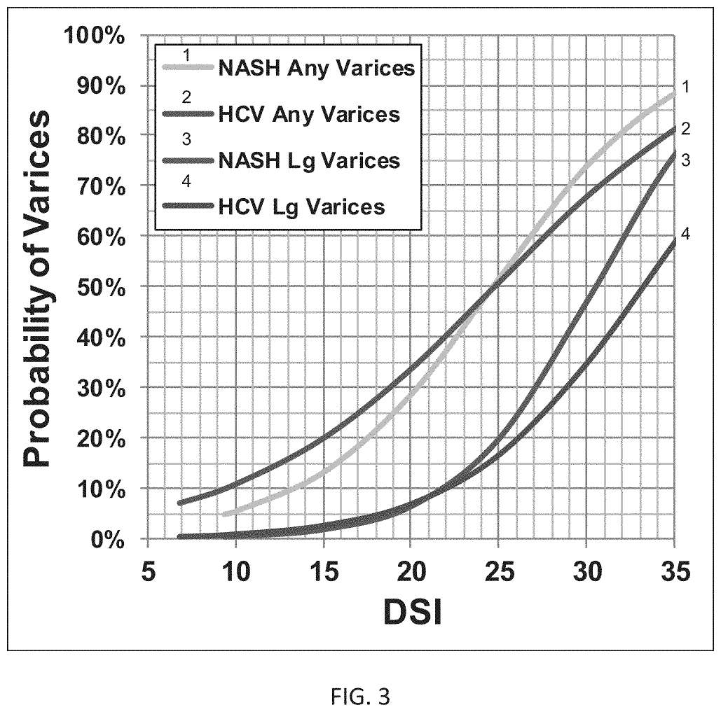

A HepQuant® test (HepQuant, LLC) can be used to determine the presence and severity of liver diseases. A HepQuant® test can be a HepQuant®-SHUNT test or a HepQuant®-STAT test. As described herein, The HepQuant®-SHUNT test uses simultaneous orally administered d4-cholate and intravenously administered 13C-cholate (i.e. intravenous (24-¹³C cholate) and oral (2,2,4,4-²H cholate) together with mass spectrometry analysis of five serum samples from the subject to accurately measure liver function and generate a disease severity index (DSI) score. HepQuant®-STAT is a simpler test that uses only orally administered d4-cholate and a single serum sample from the subject to estimate liver function (STAT). HepQuant®-SHUNT and HepQuant®-STAT have been described in U.S. Pat. Nos. 8,613,904, 8,778,299, 9,091,701, 9,417,230, 9,759,731, and 10,215,746, each of which is incorporated herein by reference in its entirety. The DSI can be used to determine the extent of liver function, and therefore the extent of liver damage/disease in the subject. Thus, the DSI score can be used to diagnose a subject with a disease. For example, a DSI greater than 16.5 can indicate that the subject will likely have biopsy-proven NASH. A DSI score can also be used to determine the percent likelihood of endoscopic finding of varices in subjects with NASH or hepatitis C virus infection, as shown in FIG. 3.

The term “administering” refers to the act of delivering an obeticholic acid composition described herein into a subject by such routes as oral, mucosal, topical, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration. The term can also refer to the frequency (e.g., daily, weekly, monthly, etc.) of providing an obeticholic acid composition described herein to a patient. Administration generally occurs after the onset of the disease, disorder, or condition, or its symptoms but, in certain instances, can occur before the onset of the disease, disorder, or condition, or its symptoms (e.g., administration for patients prone to such a disease, disorder, or condition). In certain embodiments, administration as used herein refers to oral administration.

The term “co-administration” refers to administration of two or more agents (e.g., an obeticholic acid composition described herein and another active agent described herein). The timing of co-administration depends in part of the combination and the compositions administered and can include administration at the same time, prior to, or after the administration of one or more additional therapies. An obeticholic acid composition of the instant invention can be administered alone or can be coadministered to the patient. Co-administration is meant to include simultaneous or sequential administration of an obeticholic acid composition individually or in combination (more than one compound or agent). Thus, the preparations can also be combined, when desired, with other active substances (e.g., to reduce metabolic degradation). The obeticholic acid compositions described herein can be used in combination with each other (i.e., two different obeticholic acid compositions), with other active agents known to be useful in treating a disease, or with adjunctive agents that are not effective alone, but can contribute to or enhance the efficacy of the active agent.

“Pharmacological effect” as used herein encompasses effects produced in the subject that achieve the intended purpose of a therapy. In one embodiment, a pharmacological effect means that primary indications of the subject being treated are prevented, alleviated, or reduced. For example, a pharmacological effect would be one that results in the prevention, alleviation or reduction of primary indications in a treated subject. In another embodiment, a pharmacological effect means that disorders or symptoms of the primary indications of the subject being treated are prevented, alleviated, or reduced. For example, a pharmacological effect would be one that results in the prevention or reduction of primary indications in a treated subject.

“Solvates” means solvent addition forms that contain either stoichiometric or non-stoichiometric amounts of solvent. Obeticholic acid may have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate. Additionally, the compounds of the present disclosure, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc.

A “pharmaceutical composition” is a formulation containing obeticholic acid in a form suitable for administration to a subject. In one embodiment, the pharmaceutical composition is in bulk or in unit dosage form. It is can be advantageous to formulate compositions in dosage unit form for ease of administration and uniformity of dosage. Dosage unit form, as used herein, refers to physically discrete units suited as unitary dosages for the subject to be treated; each unit containing a predetermined quantity of active reagent calculated to produce the desired therapeutic effect in association with the required pharmaceutical carrier. The specification for the dosage unit forms of the disclosure are dictated by and directly dependent on the unique characteristics of the active reagent and the particular therapeutic effect to be achieved, and the limitations inherent in the art of compounding such an active agent for the treatment of individuals.

The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient as described above.

The unit dosage form is any of a variety of forms, including, for example, a capsule, an IV bag, a tablet, a single pump on an aerosol inhaler, or a vial. The quantity obeticholic acid (e.g., a formulation of obeticholic acid, or a pharmaceutically acceptable salt, solvate, or amino acid conjugate thereof) in a unit dose of composition is an effective amount and is varied according to the particular treatment involved. One skilled in the art will appreciate that it is sometimes necessary to make routine variations to the dosage, depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, obeticholic acid is mixed under sterile conditions with a pharmaceutically acceptable carrier, and with any preservatives, buffers, or propellants that are required.

The term “flash dose” refers to obeticholic acid formulations that are rapidly dispersing dosage forms.

The term “immediate release” is defined as a release of obeticholic acid from a dosage form in a relatively brief period of time, generally up to about 60 minutes. The term “modified release” is defined to include delayed release, extended release, and pulsed release. The term “pulsed release” is defined as a series of releases of drug from a dosage form. The term “sustained release” or “extended release” is defined as continuous release of obeticholic acid from a dosage form over a prolonged period.

A “subject” or “patient” includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). In one embodiment, the patient is human. In one embodiment, the subject is human child (e.g., between about 30 kg to about 70 kg).

Patients with NASH are categorized in four groups: patients with NASH with mild fibrosis (F1) (which is largely undiagnosed and the patients may benefit from treating underlying pathology, e.g., hyperlipidemia, diabetes, obesity, lifestyle modifications); patients with NASH with moderate/severe fibrosis (F2/F3) (may benefit from treatment); and patients with NASH with cirrhosis (F4) (highest medical need population).

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A “control” as used herein refers to a baseline level determined on a patient-by-patient basis, an amount or level considered by those skilled in the art as a normal value, or any level or measure of a condition or biomarker described herein taken from a patient or population of patients at any given time for a given condition.

“Fibrosis” refers to a condition involving the development of excessive fibrous connective tissue, e.g., scar tissue, in a tissue or organ. Such generation of scar tissue may occur in response to infection, inflammation, or injury of the organ due to a disease, trauma, chemical toxicity, and so on. Fibrosis may develop in a variety of different tissues and organs, including the liver, kidney, intestine, lung, heart, etc.

“Cirrhosis” is a condition in which the liver is scarred and permanently damaged. Scar tissue replaces healthy liver tissue and prevents the liver from working normally. As cirrhosis gets worse, the liver begins to fail. Compensated cirrhosis often does not exhibit signs or symptoms related to cirrhosis, despite evidence of portal hypertension, such as esophageal or gastric varices. In contrast, decompensated cirrhosis displays symptomatic complications related to cirrhosis, including those related to hepatic insufficiency (jaundice or hepatic encephalopathy), and those related to portal hypertension (ascites or variceal hemorrhage). Prognosis and survival is markedly better in patients with compensated cirrhosis than in those with decompensated cirrhosis. In addition, the presence of decompensated cirrhosis can have major implications regarding management and prevention of cirrhosis-related complications, as well as consideration for a referral for liver transplantation evaluation.

The disclosure also comprehends isotopically-labeled obeticholic acid, or pharmaceutically acceptable salts, solvate, or amino acid conjugates thereof, which are identical to those recited in formulae of the disclosure and following, but for the fact that one or more atoms are replaced by an atom having an atomic mass or mass number different from the atomic mass or mass number most commonly found in nature. Examples of isotopes that can be incorporated into obeticholic acid, or pharmaceutically acceptable salts, solvate, or amino acid conjugates thereof include isotopes of hydrogen, carbon, nitrogen, fluorine, such as ³H, ¹¹C, ¹⁴C and ¹⁸F.

Obeticholic acid, or pharmaceutically acceptable salts, solvates, or amino acid conjugates thereof that contain the aforementioned isotopes and/or other isotopes of other atoms are within the scope of the present disclosure. Isotopically-labeled obeticholic acid, or pharmaceutically acceptable salts, solvates, or amino acid conjugates thereof, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances, isotopically labeled obeticholic acid, or pharmaceutically acceptable salts, solvates, or amino acid conjugates thereof can generally be prepared by carrying out the procedures disclosed in the Schemes and/or in the Examples of the disclosure, by substituting a readily available isotopically labeled reagent for a non-isotopically labeled reagent. In one embodiment, obeticholic acid, or pharmaceutically acceptable salts, solvates, or amino acid conjugates thereof are not isotopically labeled. In one embodiment, deuterated obeticholic acid is useful for bioanalytical assays. In another embodiment, obeticholic acid, or pharmaceutically acceptable salts, solvates, or amino acid conjugates thereof are radiolabeled.

The excipient can be any excipient present in the composition comprising obeticholic acid, or a pharmaceutically acceptable salt, ester, or amino acid conjugate thereof. Examples of excipients include, but are not limited to, calcium phosphate, microcrystalline cellulose, sodium starch glycolate and magnesium stearate, or a combination thereof. In one embodiment, the excipient can be any excipient known in the art. In another embodiment, the excipient is selected from calcium phosphate, microcrystalline cellulose, sodium starch glycolate and magnesium stearate. In yet another embodiment, the excipient is selected from microcrystalline cellulose, sodium starch glycolate and magnesium stearate. In another embodiment, the excipient is the excipient is magnesium stearate. In yet another embodiment, the excipient is microcrystalline cellulose. In a further embodiment, the excipient is sodium starch glycolate.

In another embodiment, the pharmaceutical composition comprises a therapeutically effective amount of obeticholic acid, or a pharmaceutically acceptable salt, ester, or amino acid conjugate thereof, and a pharmaceutically acceptable excipient.

In one embodiment, the pharmaceutical composition further comprises one or more pharmaceutical excipients. The excipient can be one or more selected from the group consisting of diluents, sweeteners, viscosity enhancing agents, dispersing agents, preservatives, flavoring agents and the like. One excipient can perform more than one function. In one embodiment, the one or more pharmaceutical excipients include a lubricant and/or a diluent.

Non-limiting examples of sweeteners include natural sweeteners such as sugars, e.g., fructose, glucose, sucrose, sugar alcohols such as mannitol, sorbitol or mixtures thereof and artificial sweeteners such as sodium saccharine, sodium cyclamate and aspartame. In one embodiment, the sweetener can be any sweetener known in the art. In another embodiment, the sweetener is selected from fructose, glucose, sucrose, mannitol, and sorbitol, or a combination thereof.

Dispersing agents include, but are not limited to, colloidal silicon dioxide and surfactants, wherein the surfactant is used alone or as an admixture with one or more surfactant. In one embodiment, the dispersing agent can be any dispersing agent known in the art. Combinations of colloidal silicon dioxide with one or more surfactants can also be used.

In one embodiment, the lubricant can be any lubricant known in the art. Non-limiting examples of lubricants include magnesium stearate, calcium stearate, stearic acid, glyceryl behenate, hydrogenated vegetable oil, and glycerine fumarate, and/or a combination thereof. In another embodiment, the lubricant is selected from magnesium stearate, calcium stearate, stearic acid, glyceryl behenate, hydrogenated vegetable oil, and glycerine fumarate. In another embodiment, the lubricant is calcium stearate. In yet another embodiment, the lubricant is stearic acid. In further embodiment, the lubricant is magnesium stearate.

In one embodiment, the diluent can be any diluent known in the art. Non-limiting examples of diluents include starch, pregelatinized starch, microcrystalline cellulose, calcium carbonate, dibasic calcium phosphate, tribasic calcium phosphate, calcium phosphate, lactose, dextrose, fructose, lactitol, lactose, magnesium carbonate, magnesium oxide, maltitol, maltodextrin, maltose, simethicone, sodium chloride, talc, xylitol, sorbitol, mannitol, and sucrose, and/or a combination thereof. In another embodiment, the diluent is selected from starch, pregelatinized starch, microcrystalline cellulose, calcium phosphate, lactose, sorbitol, mannitol, and sucrose. In another embodiment, the diluent is calcium phosphate. In yet another embodiment, the diluent is mannitol. In further embodiment, the diluent is microcrystalline cellulose.

In one embodiment, the pharmaceutical composition may further comprise a coating agent such as sugar-based coating agents, water-soluble film coating agents, enteric coating agents and delayed release coating agents or a coating composition comprising any combination thereof. In another embodiment, the coating agent can be any coating agent known in the art. Examples of coating agents include, but are not limited to, saccharose used alone or together with any of the agents such as talc, calcium carbonate, calcium phosphate, calcium sulphate, gelatine, gum arabic, polyvinylpyrrolidone and pullulan or any combination thereof; cellulose derivatives such as hydroxypropyl cellulose, hydroxypropyl methyl cellulose, hydroxyethyl cellulose, methyl hydroxyethyl cellulose and sodium carboxymethyl cellulose; synthetic polymers such as polyvinyl acetal diethyl amino acetate, aminoalkyl methacrylate copolymers and polyvinylpyrrolidone; polysaccharides such as pullulan; hydroxypropyl methyl cellulose phthalate; hydroxypropyl methyl cellulose acetate succinate; carboxymethyl ethyl cellulose; cellulose acetate phthalate; acrylic acid derivatives such as methacrylic acid copolymer L, methacrylic acid copolymer LD and methacrylic acid copolymer S; natural substances such as shellac; titanium dioxide; polyvinyl alcohol (e.g., Opadry®); polyethylene glycol; talc; lecithin; and/or combinations thereof. In one embodiment, the coating agent is selected from hydroxypropyl cellulose, hydroxypropyl methyl cellulose, hydroxyethyl cellulose, methyl hydroxyethyl cellulose, sodium carboxymethyl cellulose, polyvinyl acetal diethyl amino acetate, polyvinyl alcohol, polyethylene glycol, and lecithin, or a combination thereof. In another embodiment, the coating agent is Opadry® II (e.g., Opadry® II green, white, yellow, etc.).

In one embodiment, the pharmaceutically composition is in solid particle form. Any inert excipient that is commonly used as a carrier or diluent may be used in the pharmaceutical composition of the present disclosure, such as for example, a gum, a starch, a sugar, a cellulosic material, a glycolate, an acrylate, or mixtures thereof. In one embodiment, the filler/diluent is microcrystalline cellulose. The pharmaceutical composition may further comprise a disintegrating agent (e.g., sodium starch glycolate) and/or a lubricant (e.g., magnesium stearate). Also, the pharmaceutical composition may comprise one or more additives selected from a buffer, a surfactant, a solubilizing agent, a plasticizer, an emulsifier, a stabilizing agent, a viscosity increasing agent, a sweetener, a film forming agent, or any combination thereof. Furthermore, the pharmaceutical composition of the present disclosure may be in the form of controlled release of immediate release formulations.

The percentage of the active ingredient (i.e., obeticholic acid, or a pharmaceutically acceptable salt, ester, or amino acid conjugate thereof) and various excipients in the pharmaceutical composition of the present disclosure may vary. For example, the composition may comprise between about 0.1 and about 99%, between about 1-50%, between about 1-25%, or about 1-6% by weight of active ingredient. Furthermore, the composition may comprise between about 20-99%, between about 45-97%, between about 65-96%, or between about 85-95% by weight microcrystalline cellulose as a filler or diluent. Furthermore, the composition may comprise between about 1-30%, between about 1-20%, or between about 2-8% by weight sodium starch glycolate as a disintegrant. Furthermore, the composition may comprise between about 0.1-5% or about 0.5-2.0% by weight magnesium stearate as a lubricant.

In one embodiment, the pharmaceutical composition of the present disclosure is about 0.1% to about 10% by weight of active ingredient (i.e., obeticholic acid, or a pharmaceutically acceptable salt, ester, or amino acid conjugate thereof), about 0.1% to about 20% by weight of sodium starch glycolate, about 0.01% to about 8.0% by weight of magnesium stearate, and about 65% to about 99% by weight of microcrystalline cellulose. In another embodiment, the pharmaceutical composition of the present disclosure is about 0.5% to about 8% by weight of active ingredient, about 1% to about 10% by weight of sodium starch glycolate, about 0.05% to about 4.0% by weight of magnesium stearate, and about 75% to about 97% by weight of microcrystalline cellulose. In yet another embodiment, the pharmaceutical composition of the present disclosure is about 1% to about 6% by weight of active ingredient, about 2% to about 8% by weight of sodium starch glycolate, about 0.1% to about 2.0% by weight of magnesium stearate, and about 85% to about 95% by weight of microcrystalline cellulose. In one embodiment, obeticholic acid, or a pharmaceutically acceptable salt, ester, or amino acid conjugate thereof, in the pharmaceutical composition is in the form of particles.

In another aspect the present disclosure provides a method for treating or preventing a disease or condition, comprising administering an effective amount of an obeticholic acid composition of the present disclosure to a patient in need thereof.

In another aspect the present disclosure provides a method for treating NAFLD, comprising administering an effective amount of an obeticholic acid composition of the present disclosure to a patient in need thereof.

In another aspect the present disclosure provides a method for treating NASH, comprising administering an effective amount of an obeticholic acid composition of the present disclosure to a patient in need thereof.

In another aspect the present disclosure provides a method for slowing down or reversing the progression of NASH, comprising administering an effective amount of an obeticholic acid composition of the present disclosure to a patient in need thereof.

In another aspect the present disclosure provides a method for slowing down or reversing the progression of liver fibrosis, comprising administering an effective amount of an obeticholic acid composition of the present disclosure to a patient in need thereof.

In another aspect the present disclosure provides a method for slowing down or reversing the progression of cirrhosis (e.g., compensated cirrhosis due to NASH), comprising administering an effective amount of an obeticholic acid composition of the present disclosure to a patient in need thereof.

In one aspect, the present disclosure provides a method for treating a disease or condition by administering an effective amount of an obeticholic acid composition described herein to a patient in need thereof. In certain embodiments herein the effective amount refers to a titrated dosage administered during a titration period as set forth herein. In other embodiments, the effective amount refers to an adjusted or re-adjusted dosage administered after a titration period as set forth herein.

It is to be understood that the methods described herein refer generally to the obeticholic acid compositions set forth herein. In another embodiment, the obeticholic acid composition useful in the methods of treating described herein is a composition that includes microcrystalline cellulose, sodium starch glycolate, and magnesium stearate as excipients. Such a composition can be provided in a dosage form set forth herein, e.g., an oral dosage form such as a tablet or coated tablet. Thus, in certain instances, the obeticholic acid composition useful in the methods is a tablet or coated tablet for oral administration. In one embodiment, the oral dosage form of the obeticholic acid composition includes a film coating that includes one or more excipients selected from polyvinyl alcohol (part hydrolyzed), titanium dioxide, macrogol (polyethylene glycol 3350), talc, and iron oxide.

In one embodiment, the disease or condition is an FXR mediated disease or condition. Examples of the FXR mediated diseases or conditions include, but not limited to, liver diseases such as chronic liver disease, nonalcoholic fatty liver disease (NAFLD), nonalcoholic steatohepatitis (NASH), cirrhosis (e.g., compensated cirrhosis due to NASH), hepatitis C infection, alcoholic liver disease, liver damage due to progressive fibrosis, and liver fibrosis. Examples of FXR mediated diseases also include portal hypertension, bile acid diarrhea, hyperlipidemia, high LDL-cholesterol, high HDL-cholesterol, high triglycerides, and cardiovascular disease.

In another aspect, the present disclosure provides methods of treating or preventing a disease or condition described herein by administering an obeticholic acid composition described herein (e.g., obeticholic acid or a pharmaceutically acceptable salt, ester, or amino acid conjugate thereof, where the obeticholic acid or a pharmaceutically acceptable salt, ester, or amino acid conjugate thereof is in the form of particles, e.g., micronized or jet-milled particles).

NAFLD is a medical condition that is characterized by the buildup of fat (called fatty infiltration) in the liver. NAFLD is one of the most common causes of chronic liver disease, and encompasses a spectrum of conditions associated with lipid deposition in hepatocytes. It ranges from steatosis (simple fatty liver), to nonalcoholic steatohepatitis (NASH), to advanced fibrosis and cirrhosis. The disease is mostly silent and is often discovered through incidentally elevated liver enzyme levels. NAFLD is strongly associated with obesity and insulin resistance and is currently considered by many as the hepatic component of the metabolic syndrome.

NAFLD can be divided into four stages. Stage 1 is hepatic steatosis, where excess fat builds up in the liver cells but is considered harmless. There are usually no symptoms. Some stage 1 NAFLD progresses to stage 2, NASH. NASH is a more aggressive form of the condition, where the liver has become inflamed, which may indicate that liver cells have become damaged. Stage 3 is fibrosis, which is where persistent inflammation in the liver results in the generation of fibrous scar tissue around the liver cells and blood vessels. This fibrous tissue replaces some of the healthy liver tissue, but there is still enough healthy tissue for the liver to continue to function normally. In stage 4, bands of scar tissue and clumps of liver cells develop. The liver shrinks and becomes lumpy. This is known as cirrhosis. The damage caused by cirrhosis is permanent and may not be reversible (e.g., decompensated cirrhosis). Cirrhosis progresses slowly, over many years, gradually causing the liver to stop functioning (i.e., liver failure).

Various histological features can be measured to assess NAFLD and NASH, including hepatic steatosis, lobular and portal inflammation, hepatocyte ballooning, and fibrosis, through liver biopsy. Lobular inflammation consists of a mixed inflammatory cell infiltrate, composed of lymphocytes, some eosinophils, and, occasionally, a few neutrophils. Polymorphs are occasionally observed surrounding ballooned hepatocytes in a lesion known as “satellitosis”. Foci of chronic lobular inflammation are occasionally seen, while scattered lobular microgranulomas (Kupffer cell aggregates) and lipogranulomas are common. Portal chronic mononuclear cell inflammation in NASH is not uncommon. In untreated NAFLD, increased portal inflammation may serve as a marker of severe disease. Ballooned hepatocytes are enlarged, with swollen, rarefied, pale cytoplasm and, usually, show a large, hyperchromatic nucleus, often with a prominent nucleolus. Ballooning is a feature of major importance in NASH as its presence is associated in prognostic studies with more aggressive disease and high incidence of cirrhosis.

Apoptotic (acidophil) bodies, another form of hepatocyte injury and a feature of programmed cell death, may also serve as a biomarker for NASH. The number of acidophil bodies per mm² of liver tissue (acidophil body index) may be used as a complementary histological feature when diagnosis of NASH is uncertain.

Other histological lesions that may serve as biomarkers for NASH include Mallory-Denk bodies (MDB), megamitochondria, glycogenated nuclei, and iron deposition. MDB (previously called Mallory bodies or Mallory's hyaline) are eosinophilic intracytoplasmic inclusions commonly seen close to the nucleus of ballooned hepatocytes in zone 3, usually in areas of perisinusoidal fibrosis. They are composed of misfolded intermediate filaments (keratins 8 and 18), ubiquitin, heat shock proteins, and p62. MDB is correlated with increased necroinflammatory activity and a higher incidence of cirrhosis. Megamitochondria (giant mitochondria) are round or needle-shaped, eosinophilic, intracytoplasmic inclusions more commonly observed in hepatocytes with microvesicular steatosis. Ultrastructural studies have shown that these abnormal mitochondria show loss of cristae, multilamellar membranes, and paracrystalline inclusions. Megamitochondria in NASH may be the result of injury from lipid peroxidation or represent an adaptive change. Glycogenated nuclei are vacuolated nuclei usually observed in periportal hepatocytes. Their presence in biopsies with steatohepatitis is supportive of nonalcoholic etiology (obesity and/or diabetes) because they are very rarely seen in biopsies of alcoholic steatohepatitis.

Nonalcoholic steatohepatitis (NASH) is a condition that causes inflammation and accumulation of fat and fibrous (scar) tissue in the liver. Liver enzyme levels in the blood may be more elevated than the mild elevations seen with nonalcoholic fatty liver (NAFL). Although similar conditions can occur in people who abuse alcohol, NASH occurs in those who drink little to no alcohol. NASH affects 2 to 5 percent of Americans and is most frequently seen in people with one of more of the following conditions: obesity, diabetes, hyperlipidemia, insulin resistance, uses of certain medications, and exposure to toxins. NASH is an increasingly common cause of chronic liver disease worldwide and is associated with increased liver-related mortality and hepatocellular carcinoma, even in the absence of cirrhosis. NASH progresses to cirrhosis in 15-20% of affected individuals and is now one of the leading indications for liver transplantation in the United States. At present there are no approved therapies for NASH or cirrhosis (e.g., compensated cirrhosis due to NASH).

In one embodiment, the method is a method of treating NASH by administering an obeticholic acid composition described herein, optionally in a titration period as described herein. The NASH patient can be a high risk NASH patient. A “high risk NASH patient” refers to characterization by one or more of: NAS≥4; baseline fibrosis stage 2 or 3; or baseline fibrosis stage 1 with a comorbidity (type 2 diabetes, BMI≥30 kg/m2 or ALT≥60 U/L).

In one embodiment, the disease or condition is NASH. In one embodiment, the disease or condition is hyperlipidemia. In one embodiment, the disease is compensated cirrhosis due to NASH.

The present disclosure also provides a method for treating or preventing NAFLD or NASH. In one embodiment, the present disclosure provides a method for treating or preventing NAFLD or NASH that is associated with hyperlipidemia. In one embodiment, the present disclosure provides a method for treating or preventing NASH. In one embodiment, the present disclosure provides a method for treating or preventing NASH that is associated with hyperlipidemia.

In one embodiment, the subject is not suffering from a cholestatic condition.

In certain instances, the methods described herein also include assessing, monitoring, measuring, or otherwise detecting liver function. Assessing, monitoring, measuring, or otherwise detecting liver function can be performed before, during, or after a titration period described herein, or in other instances, performed during the course of any treatment described herein. Liver function can be determined by, for example, assessing, monitoring, measuring, or otherwise detecting a level of one or more liver biomarkers compared to a control. In certain instances, the control is a baseline taken from the patient before beginning treatment. In other instances, the control is preestablished baseline considered as a normal value.

In some aspects, assessing, monitoring, measuring or otherwise detecting liver function can comprise performing a HepQuant® test (HepQuant, LLC) on a subject. HepQuant®-SHUNT and HepQuant®-STAT have been described in U.S. Pat. Nos. 8,613,904, 8,778,299, 9,091,701, 9,417,230, 9,759,731, and 10,215,746, each of which is incorporated herein by reference in its entirety.

The HepQuant® test can be a HepQuant®-STAT test or a HepQuant®-SHUNT test. Thus, a HepQuant® test, such as a HepQuant®-STAT and/or a HepQuant®-SHUNT can be used to ascertain and/or quantify the efficacy of a course of treatment described herein.

The HepQuant®-SHUNT test uses simultaneous orally administered d4-cholate and intravenously administered 13C-cholate (i.e. intravenous (24-¹³C cholate) and oral (2,2,4,4-²H cholate) together with mass spectrometry analysis of five serum samples from the subject to accurately measure liver function and generate a disease severity index (DSI) score. HepQuant®-STAT is a simpler test that uses only orally administered d4-cholate and a single serum sample from the subject to estimate liver function (STAT).

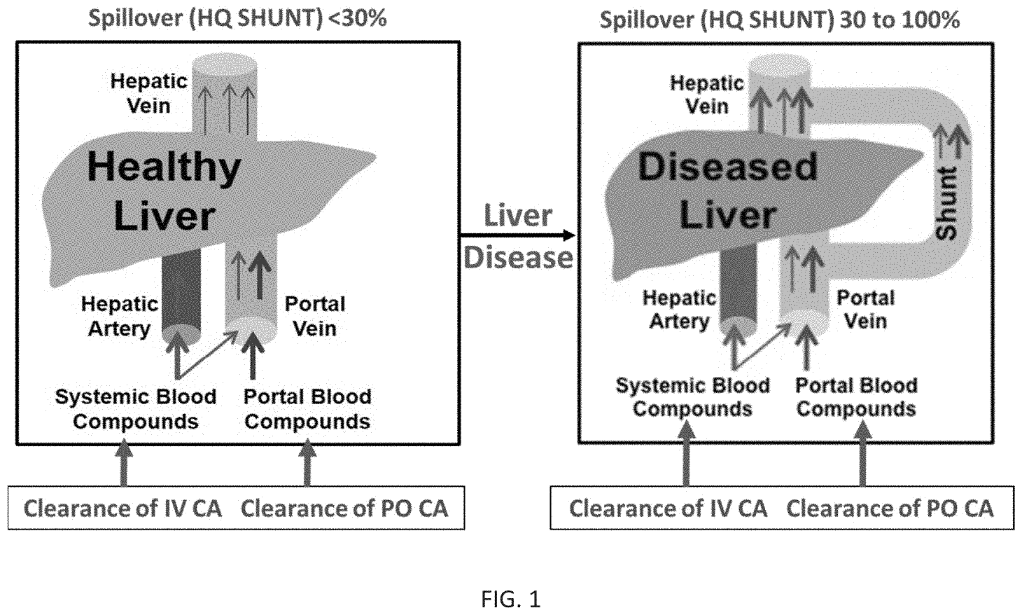

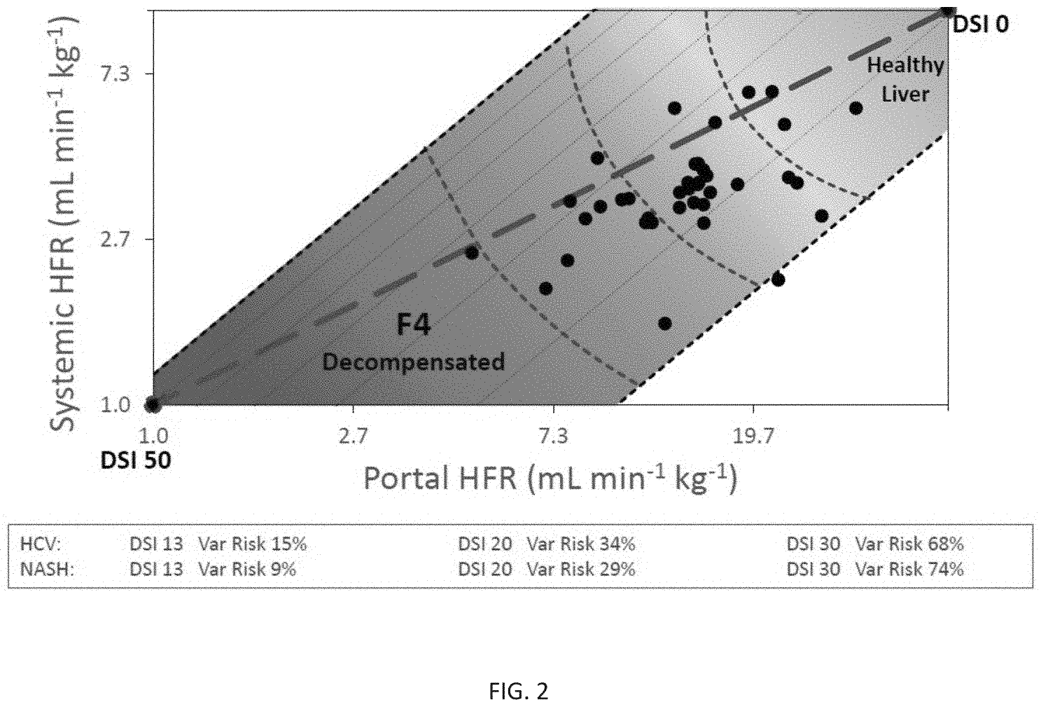

Briefly, the HepQuant®-SHUNT test allows for simultaneous measurement of the perfusion/uptake from portal circulation and perfusion/uptake from systemic circulation, as shown in FIGS. 1 and 2. In a HepQuant®-SHUNT test, a subject is orally administered 40 mg of d4-cholate and intravenously administered 20 mg of 13C-cholate. Blood samples are then isolated from the subject at 5, 20, 45, 60 and 90 minutes after cholate administration. For each sample, mass spectrometry is used to determine the Portal Hepatic Filtration Rate (Portal HFR) and Systemic Hepatic Filtration Rate (Systemic HFR). The Portal HFR is defined as the clearance of 4D-cholate (dose AUC⁻¹ kg body weight⁻¹, typically mL mini kg⁻¹) and the Systemic HFR is defined as the clearance of 13C-cholate (dose AUC⁻¹ kg body weight⁻¹, typically mL min⁻¹ kg⁻¹). The Portal HFR and Systemic HFR can then be used to calculate a Disease Severity Index (DSI) score. The DSI can be used to determine the extent of liver function, and therefore the extent of liver damage/disease in the subject. Thus, the DSI score can be used to diagnose a subject with a disease. For example, a DSI greater than 16.5 can indicate that the subject will likely have biopsy-proven NASH. A DSI score can also be used to determine the percent likelihood of endoscopic finding of varices in subjects with NASH or hepatitis C virus infection, as shown in FIG. 3. DSI scores can also be measured multiple times during a course of treatment described herein (a treatment regimen) to determine the efficacy of the course of treatment. For example, a baseline DSI score can be measured by performing a HepQuant®-SHUNT test prior to the start of the treatment regimen (i.e. prior to the first administration of the treatment regimen) and then a second DSI score can be measured at a time point during the treatment regimen. The first and second DSI scores can compared to determine a DSI difference score. For example, a reduction in DSI score from the baseline measurement can indicate that the course of treatment is beneficial as the subject's liver function is improving. Depending on the DSI difference score, the treatment regimen can be adjusted, continued or discontinued.

Thus, the present disclosure provides a method of treating a disease in a subject comprising determining a DSI score using a HepQuant® test (e.g, HepQuant®-SHUNT test); comparing the DSI score to a predetermined cutoff value; administering to the subject at least one therapeutically effective amount of obeticholic acid, or a pharmaceutically acceptable salt, ester, or amino acid conjugate thereof when the DSI score is equal to or greater than the predetermined cutoff value. The present disclosure also provides a method of treating a disease in a subject comprising determining a DSI score using a, HepQuant®-SHUNT test; comparing the DSI score to a predetermined cutoff value; administering to the subject at least one therapeutically effective amount of obeticholic acid, or a pharmaceutically acceptable salt, ester, or amino acid conjugate thereof when the DSI score is equal to or greater than the predetermined cutoff value.

In some aspects, by measuring a DSI score at multiple times during a course of treatment described herein, the treatment can be discontinued when the course of treatment provides no benefit to the subject, i.e. there is no improvement in liver function. Thus, the present disclosure also provides a method of treating a disease in a subject comprising determining a first DSI score using a HepQuant® test (e.g, HepQuant®-SHUNT test) at a time point prior to the administration of at least one therapeutically effective amount of obeticholic acid, or a pharmaceutically acceptable salt, ester, or amino acid conjugate thereof; administering to the subject at least one therapeutically effective amount of obeticholic acid, or a pharmaceutically acceptable salt, ester, or amino acid conjugate thereof; determining at least a second DSI score using a HepQuant® test (e.g, HepQuant®-SHUNT test) test at at least one time point after the administration of the at least one therapeutically effective amount of obeticholic acid, or a pharmaceutically acceptable salt, ester, or amino acid conjugate thereof; determining a DSI difference score by calculating the difference between the at least second DSI score and the first DSI score; comparing the DSI difference score to a predetermined cutoff value; and discontinuing administration of the at least one therapeutically effective amount of obeticholic acid when the DSI difference score is less than or equal to the predetermined cutoff value.

The present disclosure also provides a method of treating a disease in a subject comprising determining a first DSI score using a HepQuant®-SHUNT test at a time point prior to the administration of at least one therapeutically effective amount of obeticholic acid, or a pharmaceutically acceptable salt, ester, or amino acid conjugate thereof; administering to the subject at least one therapeutically effective amount of obeticholic acid, or a pharmaceutically acceptable salt, ester, or amino acid conjugate thereof; determining at least a second DSI score using a HepQuant®-SHUNT test at at least one time point after the administration of the at least one therapeutically effective amount of obeticholic acid, or a pharmaceutically acceptable salt, ester, or amino acid conjugate thereof; determining a DSI difference score by calculating the difference between the at least second DSI score and the first DSI score; comparing the DSI difference score to a predetermined cutoff value; and discontinuing administration of the at least one therapeutically effective amount of obeticholic acid when the DSI difference score is less than or equal to the predetermined cutoff value.

In some aspects, by measuring a DSI score at multiple times during a course of treatment described herein and calculating a DSI difference score, the treatment can be continued when the course of treatment provides a benefit to the subject, i.e. the treatment is causing an improvement in liver function. Thus, the present disclosure also provides a method of treating a disease in a subject comprising determining a first DSI score using a HepQuant® test (e.g, HepQuant®-SHUNT test) at a time point prior to the administration of at least one therapeutically effective amount of obeticholic acid, or a pharmaceutically acceptable salt, ester, or amino acid conjugate thereof; administering to the subject at least one therapeutically effective amount of obeticholic acid, or a pharmaceutically acceptable salt, ester, or amino acid conjugate thereof; determining at least a second DSI score using a HepQuant® test (e.g, HepQuant®-SHUNT test) at at least one time point after the administration of the at least one therapeutically effective amount of obeticholic acid, or a pharmaceutically acceptable salt, ester, or amino acid conjugate thereof; determining a DSI difference score by calculating the difference between the at least second DSI score and the first DSI score; comparing the DSI difference score to a predetermined cutoff value; and continuing administration of the at least one therapeutically effective amount of obeticholic acid when the DSI difference score is greater than or equal to the predetermined cutoff value.

The present disclosure also provides a method of treating a disease in a subject comprising determining a first DSI score using a HepQuant®-SHUNT test at a time point prior to the administration of at least one therapeutically effective amount of obeticholic acid, or a pharmaceutically acceptable salt, ester, or amino acid conjugate thereof; administering to the subject at least one therapeutically effective amount of obeticholic acid, or a pharmaceutically acceptable salt, ester, or amino acid conjugate thereof; determining at least a second DSI score using a HepQuant®-SHUNT test at at least one time point after the administration of the at least one therapeutically effective amount of obeticholic acid, or a pharmaceutically acceptable salt, ester, or amino acid conjugate thereof; determining a DSI difference score by calculating the difference between the at least second DSI score and the first DSI score; comparing the DSI difference score to a predetermined cutoff value; and continuing administration of the at least one therapeutically effective amount of obeticholic acid when the DSI difference score is greater than or equal to the predetermined cutoff value.

In some aspects, a DSI difference score that is greater than or equal to 2 (i.e. a decrease in the DSI scores of 2 or more over the course of treatment) is considered to be clinically meaningful, based on reducing the probability of any varices from approximately 30% to 20% for subjects with a baseline DSI of 20.

In some aspects, by measuring a DIS score at multiple times during a course of treatment described herein and calculating a DSI difference score, aspects of the course of treatment can be adjusted. For example, the therapeutically effective amount of obeticholic acid, or a pharmaceutically acceptable salt, ester, or amino acid conjugate thereof can be increased or decreased accordingly based on the DSI difference score. Thus, the present disclosure also provides a method of treating a disease in a subject comprising determining a first DSI score using a HepQuant® test (e.g, HepQuant®-SHUNT test) at a time point prior to the administration of at least one first therapeutically effective amount of obeticholic acid, or a pharmaceutically acceptable salt, ester, or amino acid conjugate thereof; administering to the subject at least one first therapeutically effective amount of obeticholic acid, or a pharmaceutically acceptable salt, ester, or amino acid conjugate thereof; determining at least a second DSI score using a HepQuant® test (e.g, HepQuant®-SHUNT test) at at least one time point after the administration of the at least one first therapeutically effective amount of obeticholic acid, or a pharmaceutically acceptable salt, ester, or amino acid conjugate thereof; determining a DSI difference score by calculating the difference between the at least second DSI score and the first DSI score; comparing the DSI difference score to a predetermined cutoff value; administering to the subject at least one second therapeutically effective amount of obeticholic acid, or a pharmaceutically acceptable salt, ester, or amino acid conjugate thereof when the DSI difference score is greater than or equal to the predetermined cutoff value.

The present disclosure also provides a method of treating a disease in a subject comprising determining a first DSI score using a HepQuant®-SHUNT test at a time point prior to the administration of at least one first therapeutically effective amount of obeticholic acid, or a pharmaceutically acceptable salt, ester, or amino acid conjugate thereof; administering to the subject at least one first therapeutically effective amount of obeticholic acid, or a pharmaceutically acceptable salt, ester, or amino acid conjugate thereof; determining at least a second DSI score using a HepQuant®-SHUNT test at at least one time point after the administration of the at least one first therapeutically effective amount of obeticholic acid, or a pharmaceutically acceptable salt, ester, or amino acid conjugate thereof; determining a DSI difference score by calculating the difference between the at least second DSI score and the first DSI score; comparing the DSI difference score to a predetermined cutoff value; administering to the subject at least one second therapeutically effective amount of obeticholic acid, or a pharmaceutically acceptable salt, ester, or amino acid conjugate thereof when the DSI difference score is greater than or equal to the predetermined cutoff value.

Alternatively, the present disclosure also provides a method of treating a disease in a subject comprising determining a first DSI score using a HepQuant® test (e.g, HepQuant®-SHUNT test) at a time point prior to the administration of at least one first therapeutically effective amount of obeticholic acid, or a pharmaceutically acceptable salt, ester, or amino acid conjugate thereof; administering to the subject at least one first therapeutically effective amount of obeticholic acid, or a pharmaceutically acceptable salt, ester, or amino acid conjugate thereof; determining at least a second DSI score using a HepQuant® test (e.g, HepQuant®-SHUNT test) at at least one time point after the administration of the at least one first therapeutically effective amount of obeticholic acid, or a pharmaceutically acceptable salt, ester, or amino acid conjugate thereof; determining a DSI difference score by calculating the difference between the at least second DSI score and the first DSI score; comparing the DSI difference score to a predetermined cutoff value; administering to the subject at least one second therapeutically effective amount of obeticholic acid, or a pharmaceutically acceptable salt, ester, or amino acid conjugate thereof when the DSI difference score is less than or equal to the predetermined cutoff value.

The present disclosure also provides a method of treating a disease in a subject comprising determining a first DSI score using a HepQuant®-SHUNT test at a time point prior to the administration of at least one first therapeutically effective amount of obeticholic acid, or a pharmaceutically acceptable salt, ester, or amino acid conjugate thereof; administering to the subject at least one first therapeutically effective amount of obeticholic acid, or a pharmaceutically acceptable salt, ester, or amino acid conjugate thereof; determining at least a second DSI score using a HepQuant®-SHUNT test at at least one time point after the administration of the at least one first therapeutically effective amount of obeticholic acid, or a pharmaceutically acceptable salt, ester, or amino acid conjugate thereof; determining a DSI difference score by calculating the difference between the at least second DSI score and the first DSI score; comparing the DSI difference score to a predetermined cutoff value; administering to the subject at least one second therapeutically effective amount of obeticholic acid, or a pharmaceutically acceptable salt, ester, or amino acid conjugate thereof when the DSI difference score is less than or equal to the predetermined cutoff value.

In some aspects of the preceding methods, the at least one first therapeutically effective amount can be less than the at least one second therapeutically effective amount (increasing the amount administered to the subject based on the DSI difference score). Alternatively, the at least one first therapeutically effective amount can be more than the at least one second therapeutically effective amount (decreasing the amount administered to the subject based on the DSI difference score). Alternatively still, the at least one first therapeutically effective amount can be the same as the at least one second therapeutically effective amount (no change in the amount administered to the subject based on the DSI difference score).

In some aspects, the preceding methods can comprise determining at least a third, or at least a fourth, or at least a fifth, or at least a sixth, or at least a seventh, or at least an eighth, or at least a ninth, or at least a tenth DSI score at at least two, at least three, at least four, at least five, at least six, at least seven, at least eight, at least nine or at least ten time points after the administration of at least one first therapeutically effective amount of obeticholic acid, or a pharmaceutically acceptable salt, ester, or amino acid conjugate thereof.

In some aspect of the preceding methods, a time point prior to the administration of at least one first therapeutically effective amount of obeticholic acid, or a pharmaceutically acceptable salt, ester, or amino acid conjugate thereof can be at least one hour, at least two hours, at least three hours, at least four hours, at least five hours, at least six hours, at least seven hours, at least eight hour, at least nine hours, at least ten hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least one day, at least two days, at least three days, at least four days, at least five days, at least six days, at least one week, at least two weeks, at least three weeks, at least one month or at least two months before the administration of the at least one first therapeutically effective amount of obeticholic acid, or a pharmaceutically acceptable salt, ester, or amino acid conjugate thereof.

In some aspects of the preceding methods, the at least one time point after the administration of the at least one first therapeutically effective amount of obeticholic acid, or a pharmaceutically acceptable salt, ester, or amino acid conjugate thereof can be at least one day, at least two days, at least three days, at least four days, at least five days, at least six days, at least seven days, at least eight days, at least nine days, at least ten days, at least 11 days, at least 12 days, at least 13 days, at least two weeks, at least three weeks, at least four weeks, at least five weeks, at least six weeks, at least seven weeks, at least eight weeks, at least nine weeks, at least ten weeks, at least eleven weeks, at least 12 weeks, at least 85 days, at least 13 weeks, at least 14 weeks, at least 15 weeks, at least 16 weeks, at least 17 weeks, at least 18 weeks, at least 19 weeks or at least 20 weeks after the administration of the at least one first therapeutically effective amount of obeticholic acid, or a pharmaceutically acceptable salt, ester, or amino acid conjugate thereof.

In some aspects of the methods of the present disclosure comprising a HepQuant® test, the subject must be able to ingest liquid by mouth and/or have venous access to support a peripheral IV and for example, 6 blood draws. In some aspects of the methods of the present disclosure comprising a HepQuant® test, the subject cannot have had an initial dose of obeticholic acid, or a pharmaceutically acceptable salt, ester, or amino acid conjugate thereof; cannot be hypersensitive to eggs, albumin preparations, any ingredient in the HepQuant® test compositions or any components of the HepQuant® test container; cannot have intercurrent medical or surgical illness (e.g. acute MI, acute cerebral hemorrhage, sepsis); cannot have had extensive resection of large segments of small intestine (short gut); and/or cannot have severe gastroparesis.

Values for measure or detection of liver function biomarkers and controls can be expressed as a comparison to Upper Limit of Normal (ULN).

Liver biomarkers can be used to ascertain, quantify the efficacy of the course of treatment with an obeticholic acid composition described herein. In other instances, liver biomarkers described herein can be used to ascertain, quantify liver function during the course of treatment with an obeticholic acid composition described herein. Liver biomarkers can also be used to predict whether a patient or patient population will be susceptible to treatment with an obeticholic acid composition described herein. In one embodiment, the liver biomarkers include assessing, monitoring, measuring or otherwise detecting an amount or level of aspartate transaminase (AST), alanine transaminase (ALT), alkaline phosphatase (ALP), bilirubin, glycine conjugated obeticholic acid, taurine conjugated obeticholic acid, a bile acid, a bile acid glycine conjugate, or a bile acid taurine conjugate. For example, the liver biomarker assessed, monitored, measured, or detected can be ALP.

The ALP level can be a measure of ULN. In one embodiment, a patient before treatment can have an ALP level of at least 1.1×ULN to at least 20×ULN; at least 1.1×ULN to at least 15×ULN; at least 1.1×ULN to at least 12×ULN; at least 1.1×ULN to at least 10×ULN; at least 1.1×ULN to at least 8×ULN; at least 1.1×ULN to at least 6×ULN; at least 1.1×ULN to at least 5×ULN; at least 1.1×ULN to at least 4×ULN; at least 1.1×ULN to at least 3×ULN; or at least 1.1×ULN to at least 2×ULN.

A patient before a treatment described herein can have an ALP level of about 1.5×ULN to about 20×ULN; about 1.5×ULN to about 15×ULN; about 1.5×ULN to about 10 ULN; about 1.5×ULN to about 5×ULN; or about 1.5×ULN to about 3×ULN. A patient before treatment can have an ALP level before a treatment described herein of about 1.5×, 2×, 3×, 4×, 5×, 8×, 10×, 15×, or 20×ULN.

A patient before treatment can have an ALP level before a treatment described herein of greater than about 1.5×, 2×, 3×, 4×, 5×, 8×, 10×, 15×, or 20×ULN. In one embodiment, a patient has an ALP level of about 1.5×ULN. In one embodiment, a patient has an ALP level of about 2×ULN. In one embodiment, a patient has a ALP level of about 5×ULN. In one embodiment, a patient has an ALP level of about 10×ULN. In one embodiment, a patient has an ALP level of about 15×ULN. In one embodiment, a patient has an ALP level greater than about 1.5×ULN. In one embodiment, a patient has an ALP level greater than about 2×ULN. In one embodiment, a patient has a ALP level greater than about 5×ULN. In one embodiment, a patient has an ALP level greater than about 10×ULN.

In one embodiment, a patient has an ALP level greater than about 15×ULN.

In another example, the liver biomarker assessed, monitored, measured, or detected can be bilirubin. The bilirubin level can be a measure of ULN. In one embodiment, a patient before treatment can have a bilirubin level of at least 1.1×ULN to at least 20×ULN; at least 1.1×ULN to at least 15×ULN; at least 1.1×ULN to at least 12×ULN; at least 1.1×ULN to at least 10×ULN; at least 1.1×ULN to at least 8×ULN; at least 1.1×ULN to at least 6×ULN; at least 1.1×ULN to at least 5×ULN; at least 1.1×ULN to at least 4×ULN; at least 1.1×ULN to at least 3×ULN; or at least 1.1×ULN to at least 2×ULN.

A patient before a treatment described herein can have a bilirubin level of about 1.5×ULN to about 20×ULN; about 1.5×ULN to about 15×ULN; about 1.5×ULN to about 10 ULN; about 1.5×ULN to about 5×ULN; or about 1.5×ULN to about 3×ULN. In another example a patient before a treatment described herein can have a bilirubin level of about 2×ULN to about 20×ULN; about 2×ULN to about 15×ULN; about 2×ULN to about 10 ULN; about 2×ULN to about 5×ULN; or about 2×ULN to about 3×ULN. In another example a patient before a treatment described herein can have a bilirubin level of greater than about 2×ULN to greater than about 20×ULN; greater than about 2×ULN to greater than about 15×ULN; greater than about 2×ULN to greater than about 10 ULN; greater than about 2×ULN to greater than about 5×ULN; or greater than about 2×ULN to greater than about 3×ULN.

A patient before treatment can have a bilirubin level before a treatment described herein of about 1.5×, 2×, 3×, 4×, 5×, 8×, 10×, 15×, or 20×ULN. A patient before treatment can have a bilirubin level before a treatment described herein of greater than about 1.5×, 2×, 3×, 4×, 5×, 8×, 10×, 15×, or 20×ULN. In one embodiment, a patient has a bilirubin level greater than about 2×ULN. In one embodiment, a patient has a bilirubin level greater than about 5×ULN. In one embodiment, a patient has a bilirubin level greater than about 10×ULN. In one embodiment, a patient has a bilirubin level greater than about 15×ULN. In one embodiment, a patient has a bilirubin level less than about 2×ULN. In one embodiment, a patient has a bilirubin level less than about 5×ULN. In one embodiment, a patient has a bilirubin level less than about 10×ULN. In one embodiment, a patient has a bilirubin level less than about 15×ULN.

In some instances it can be useful to assess, monitor, measure, or detect ALP and bilirubin to assess, monitor, measure, or otherwise detect liver function or changes in liver function during treatment with an obeticholic acid composition described herein. In certain instances, a patient has an ALP level as provided above (e.g., about 1.5×ULN to about 10×ULN) and a bilirubin level as provided above (e.g., less than about 5×ULN). In one embodiment, the patient has an ALP level between about 1.5×ULN to about 10×ULN and a bilirubin level less than about 2×ULN.

Treatment with obeticholic acid composition described herein can reduce the levels of ALP and/or bilirubin in a patient described herein. For example, treatment of a disease or condition described herein with an obeticholic acid composition described herein can reduce the level of ALP by 2, 4, 5, 6, 8, 9, 10, 12, 15, 18, 20, 21, 22, 23, 24, 25, 26, 27, 28, 29, 30, 35, 40, 45, 50, 55, 60, 65, 70, 75, 80, 85, 88, 90, 92, 94, 96, 97, 98, 99, 99.2, 99.4, 99.6, 99.7, 99.8, 99.9, or 100%. In another example, the level of ALP can be reduced by at least 100%, at least 125%, at least 150%, at least 175%, at least 200%, at least 225%, at least 250% or at least 300%.

In another example, the level of ALP can be reduced by about 5% to about 50%; about 10% to about 55%; about 10% to about 45%; about 10% to about 40%; about 10% to about 33%, about 10% to about 30%; about 15% to about 30%; about 15% to about 25%; about 20% to about 50%, about 20% to about 40%; about 20% to about 35%; about 20% to about 30%; 20% to about 27%; or about 20% to about 27%. In another example, the level of ALP can be reduced by at least 50%. The level of ALP can be reduced by at least 40%. The level of ALP can be reduced by at least 35%. The level of ALP can be reduced by at least 30%. The level of ALP can be reduced by at least 27%. The level of ALP can be reduced by at least 25%. The level of ALP can be reduced by at least 20%.

The reduction of ALP levels can be represented by the fold change over ULN. For example, treatment with an obeticholic acid described herein can reduce the ALP level of a patient described herein to less than about 5×ULN; less than about 4×ULN, less than about 3×ULN, less than about 2×ULN, less than about 1.7×ULN, less than about 1.5×ULN, less than about 1.25×ULN, or less than about ULN.

In another example, the ALP level is reduced by at least 1, 2, 3, 4, 5, 6, 7, 8, 9, 10, 15, 20, 25, 30, 40, or 50 fold compared to a baseline value. For example, the ALP level after treatment with an obeticholic acid composition described herein can be reduced by 1, 1.2, 1.4, 1.6, 1.8, or 2 fold, including intervening values therein, compared to a baseline value. In another example, the ALP level can be reduced by 2, 2.2, 2.4, 2.6, 2.8, or 3 fold, including intervening values therein, compared to a baseline value. In another example, the ALP level can be reduced 3, 4, or 5 fold, including intervening values therein, compared to a baseline value. In another example, the ALP level can be reduced 5, 7, 9, or 10 fold, including intervening values therein, compared to a baseline value. In another example, the ALP level can be reduced 10, 12, 15, or 20 fold, including intervening values therein, compared to a baseline value.

Treatment of a disease or condition described herein with an obeticholic acid composition described herein can reduce the level of bilirubin by 2, 4, 5, 6, 8, 9, 10, 12, 15, 18, 20, 21, 22, 23, 24, 25, 26, 27, 28, 29, 30, 35, 4-, 45, 50, 55, 60, 65, 70, 75, 80, 85, 88, 90, 92, 94, 96, 97, 98, 99, 99.2, 99.4, 99.6, 99.7, 99.8, 99.9, or 100%. In another example, the level of bilirubin can be reduced by at least 100%, at least 125%, at least 150%, at least 175%, at least 200%, at least 225%, at least 250% or at least 300%.

In another example, the level of bilirubin can be reduced by about 5% to about 50%; about 10% to about 55%; about 10% to about 45%; about 10% to about 40%; about 10% to about 33%, about 10% to about 30%; about 15% to about 30%; about 15% to about 25%; about 20% to about 50%, about 20% to about 40%; about 20% to about 35%; about 20% to about 30%; 20% to about 27%; or about 20% to about 27%. In another example, the level of bilirubin can be reduced by at least 50%. The level of bilirubin can be reduced by at least 40%. The level of bilirubin can be reduced by at least 35%. The level of bilirubin can be reduced by at least 30%. The level of bilirubin can be reduced by at least 27%. The level of bilirubin can be reduced by at least 25%. The level of bilirubin can be reduced by at least 20%.

The reduction of bilirubin levels can be represented by the fold change over ULN. For example, treatment with an obeticholic acid described herein can reduce the bilirubin level of a patient described herein to less than about 5×ULN; less than about 4×ULN, less than about 3×ULN, less than about 2×ULN, less than about 1.7×ULN, less than about 1.5×ULN, less than about 1.25×ULN, or less than about ULN.

In another example, the bilirubin level is reduced by at least 1, 2, 3, 4, 5, 6, 7, 8, 9, 10, 15, 20, 25, 30, 40, or 50 fold compared to a baseline value. For example, the bilirubin level after treatment with an obeticholic acid composition described herein can be reduced by 1, 1.2, 1.4, 1.6, 1.8, or 2 fold, including intervening values therein, compared to a baseline value. In another example, the bilirubin level can be reduced by 2, 2.2, 2.4, 2.6, 2.8, or 3 fold, including intervening values therein, compared to a baseline value. In another example, the bilirubin level can be reduced 3, 4, or 5 fold, including intervening values therein, compared to a baseline value. In another example, the bilirubin level can be reduced 5, 7, 9, or 10 fold, including intervening values therein, compared to a baseline value. In another example, the bilirubin level can be reduced 10, 12, 15, or 20 fold, including intervening values therein, compared to a baseline value.

In another embodiment, one or more biomarkers can stratify a patient population undergoing or who will undergo treatment with an obeticholic acid composition described herein. For example, a NASH patient can be stratified for the risk of cirrhosis.

In yet another embodiment, liver biomarkers useful for detection can include metabolites and bile acids. For example, assessing, monitoring, measuring, or otherwise detecting levels of glycine and taurine conjugates of obeticholic acid can be useful for measuring efficacy of a treatment regimen described herein. For example, assessing, monitoring, measuring, or otherwise detecting levels or detecting plasma levels of bile acids including cholic acid, chenodeoxycholic acid, deoxycholic acid, lithocholic acid, and urosodeoxycholic acid, including glycine and taurine conjugates thereof, and optionally comparing the levels to a control, can be useful for measuring efficacy of a treatment regimen described herein.

In still other embodiments, calculating an AST to platelet index (APRI) can be useful for assessing, monitoring, measuring, or otherwise detecting liver function (including changes thereof). The obeticholic acid compositions described herein can reduce the APRI of a patient described herein. In certain instances, monitoring or measuring the APRI can be used to determine efficacy of treatment with an obeticholic acid composition described herein. In some embodiments, a reduction in APRI is observed in a patient (e.g., a NASH patient) after administration of an obeticholic acid composition described herein. For example, the APRI may be reduced by about 5% to about 50% in patients treated with obeticholic acid relative to baseline levels measured before dose administration. The reduction may be up to about 5%, 10%, 15%, 20%, 25%, 30%, 35%, 40%, 45% or 50%.

Further provided herein is a method for treating NAFLD or NASH in a patient in need thereof by administering a starting dose of an obeticholic acid composition described herein in a titration period. The method includes assessing liver function of the patient before, during, and after said titration period by either calculating an APRI score for said patient; or by measuring the level of one or more liver biomarker selected from ALP, bilirubin, AST, ALT, glycine conjugated obeticholic acid, taurine conjugated obeticholic acid, a bile acid, a bile acid glycine conjugate, or a bile acid taurine conjugate, where a reduced APRI score compared to a control or a reduced level of the one or more liver biomarkers compared to a control indicates non-impaired liver function. The method further includes assessing tolerance of the patient to the starting dose by grading the severity of one or more adverse effects, if present, and administering an adjusted dose of the obeticholic acid composition, where the adjusted dose includes an amount equal to or greater than an amount of the starting dose.

The starting dose, adjusted dose, and titration period are as described below. For example, the starting dose can be about 1 mg to about 50 mg, about 1 mg to about 40 mg, about 1 mg to about 30 mg, about 1 mg to about 25 mg, about 1 mg to about 20 mg, about 1 mg to about 10 mg, about 1 mg to about 5 mg, about 2 mg to about 50 mg, about 2 mg to about 40 mg, about 2 mg to about 30 mg, about 2 mg to about 25 mg, about 2 mg to about 20 mg, about 2 mg to about 10 mg, about 2 mg to about 5 mg, about 3 mg to about 50 mg, about 3 mg to about 40 mg, about 3 mg to about 30 mg, about 3 mg to about 25 mg, about 3 mg to about 20 mg, about 3 mg to about 10 mg, about 3 mg to about 5 mg, about 4 mg to about 50 mg, about 4 mg to about 40 mg, about 4 mg to about 30 mg, about 4 mg to about 25 mg, about 4 mg to about 20 mg, about 4 mg to about 10 mg, about 4 mg to about 5 mg, about 5 mg to about 50 mg, about 5 mg to about 40 mg, about 5 mg to about 30 mg, about 5 mg to about 25 mg, about 5 mg to about 20 mg, or about 5 mg to about 10 mg. For example, the starting dose can be about 1, 2, 3, 4, 5, 6, 7, 8, 9, 10, 11, 12, 13, 14, 15, 16, 17, 18, 19, 20, 21, 22, 23, 24, 25, 30, 35, 40, 45, or 50 mg. For example, the adjusted dose can be about 1 mg to about 50 mg, about 1 mg to about 40 mg, about 1 mg to about 30 mg, about 1 mg to about 25 mg, about 1 mg to about 20 mg, about 1 mg to about 10 mg, about 1 mg to about 5 mg, about 2 mg to about 50 mg, about 2 mg to about 40 mg, about 2 mg to about 30 mg, about 2 mg to about 25 mg, about 2 mg to about 20 mg, about 2 mg to about 10 mg, about 2 mg to about 5 mg, about 3 mg to about 50 mg, about 3 mg to about 40 mg, about 3 mg to about 30 mg, about 3 mg to about 25 mg, about 3 mg to about 20 mg, about 3 mg to about 10 mg, about 3 mg to about 5 mg, about 4 mg to about 50 mg, about 4 mg to about 40 mg, about 4 mg to about 30 mg, about 4 mg to about 25 mg, about 4 mg to about 20 mg, about 4 mg to about 10 mg, about 4 mg to about 5 mg, about 5 mg to about 50 mg, about 5 mg to about 40 mg, about 5 mg to about 30 mg, about 5 mg to about 25 mg, about 5 mg to about 20 mg, or about 5 mg to about 10 mg. For example, the adjusted dose can be about 1, 2, 3, 4, 5, 6, 7, 8, 9, 10, 11, 12, 13, 14, 15, 16, 17, 18, 19, 20, 21, 22, 23, 24, 25, 30, 35, 40, 45, or 50 mg. For example, the titration period can be a time of about 1 to about 6 months, e.g., 1 month, 2 months, 3 months, 4 months, 5 months, or 6 months.

Also provided herein are methods to reduce or eliminate rejection failure of a liver transplant by administering an effective amount of an obeticholic acid composition described above. In certain instances, administration of an obeticholic acid composition described herein reduces expression or levels of ALP and/or bilirubin. In one embodiment, administration of an obeticholic acid composition described herein reduces ALP and bilirubin levels, thereby reducing transplant complications or rejection.

In one aspect, obeticholic acid may mediate its action primarily via FXR agonism, wherein FGF-19 released from gut enterocytes (in response to FXR agonist) into portal circulation down regulates endogenous bile acid synthesis in the liver. The present disclosure comprehends a method of measuring FXR agonist activity by, for example, measuring release of FGF-19 into the bloodstream or circulation of a patient administered with OCA. Levels of FGF-19 may be measured by methods known in the art, such as those described herein.

Obeticholic acid administration may lead to a significant and a dose-dependent increase in the levels of FGF-19 and in some embodiments, a decrease in the levels of endogenous bile acids and C4 (a bile acid precursor). In some embodiments, a significant increase in FGF-19 levels may be observed from baseline to month 3, month 6 and month 12 after dose administration. In some examples, the FGF-19 levels may increase from about 5% to about 200%. In specific embodiments, the levels may increase by about 5%, 10%, 15%, 20%, 25%, 30%, 35%, 40%, 45%, 50%, 55%, 60%, 65%, 70%, 75%, 80%, 85%, 90%, 95% or 100%.

In some embodiments, the plasma levels of FGF-19, a marker of FXR activation, are determined using a qualified method and a validated method using an enzyme-linked immunosorbent assay (ELISA) method. The plasma concentrations of FGF-19 may be quantitated at predose and after administration of dose.

In some examples, a monoclonal antibody specific for FGF-19 is pre-coated onto a microplate. Standards, quality controls and samples are pipetted into the wells and any FGF-19 present is bound by the immobilized antibody. After washing away any unbound substances, an enzyme-linked polyclonal antibody specific for FGF-19 is added to the wells. Following a wash to remove any unbound antibody-enzyme reagent, a substrate solution is added to the wells and color develops in the proportion to the amount of FGF-19 bound in the initial step. The color development is stopped and the intensity of the color is measured. The calibration range of the method is 15.625 pg/ml to 1000 pg/ml for FGF-19 using a 100 μl aliquot of standard curve, quality control and sample. In some embodiments, no minimum required dilution is used. In other embodiments, samples may be subjected to a 3× minimum required dilution.

In another aspect of the disclosure is a method of treating a solid-tumor cancer by administering an effective amount of an obeticholic acid composition as described herein. In another aspect, such methods include treating liver cancer by administering an obeticholic acid composition as described herein. Liver cancer includes hepatocellular carcinoma (HCC) and bile duct cancer (cholangiocarcinoma). Risk factors for HCC include chronic infection with hepatitis B or C and cirrhosis of the liver. In one embodiment is a method of treating HCC by administering an effective amount of an obeticholic acid composition as described herein. In one embodiment is a method of treating colorectal cancer by administering an effective amount of an obeticholic acid composition as described herein. In another embodiment is a method of treating gastric cancer by administering an effective amount of an obeticholic acid composition as described herein. In another embodiment is a method of treating liver cancer by administering an effective amount of an obeticholic acid composition as described herein. In still another embodiment is a method of treating renal cancer by administering an effective amount of an obeticholic acid composition as described herein. In still another embodiment is a method of treating pancreatic cancer by administering an effective amount of an obeticholic acid composition as described herein. It is understood that the treatment of a cancer described herein can be performed by administering an effective amount of an obeticholic acid composition described herein in combination with one or more anticancer agents, such as those described herein. In some embodiments, the effective amount administered is a starting dose as described herein.

In another aspect, the present disclosure also provides a method for inhibiting or reversing fibrosis, comprising administering a therapeutically effective amount of the composition of the present disclosure to a subject in need thereof.

In one embodiment, the subject is suffering from a condition selected from the group consisting of cancers, such as, e.g., cancers as described herein, including primary liver and biliary cancer, metastatic cancer, sepsis, chronic total parenteral nutrition, cystic fibrosis, and granulomatous liver disease. In embodiments, the fibrosis to be inhibited occurs in an organ where FXR is expressed.

In one embodiment, the fibrosis is selected from the group consisting of liver fibrosis, kidney fibrosis, and intestinal fibrosis.

In one embodiment, the subject has liver fibrosis associated with a disease selected from the group consisting of hepatitis B; hepatitis C; parasitic liver diseases; post-transplant bacterial, viral and fungal infections; alcoholic liver disease (ALD); non-alcoholic fatty liver disease (NAFLD); non-alcoholic steatohepatitis (NASH); liver diseases induced by methotrexate, isoniazid, oxyphenistatin, methyldopa, chlorpromazine, tolbutamide, or amiodarone; autoimmune hepatitis; sarcoidosis; Wilson's disease; hemochromatosis; Gaucher's disease; types III, IV, VI, IX and X glycogen storage diseases; ai-antitrypsin deficiency; Zellweger syndrome; tyrosinemia; fructosemia; galactosemia; vascular derangement associated with Budd-Chiari syndrome, veno-occlusive disease, or portal vein thrombosis; and congenital hepatic fibrosis.

In another embodiment, the subject has intestinal fibrosis associated with a disease selected from the group consisting of Crohn's disease, ulcerative colitis, post-radiation colitis, and microscopic colitis.

In another embodiment, the subject has renal fibrosis associated with a disease selected from the group consisting of diabetic nephropathy, hypertensive nephrosclerosis, chronic glomerulonephritis, chronic transplant glomerulopathy, chronic interstitial nephritis, and polycystic kidney disease.

In another aspect, the present disclosure also provides a method for treating or preventing all forms of conditions related to elevated lipid levels. In one embodiment, the condition is hyperlipidemia where it is associated with a condition selected from non-alcohol-induced steatohepatitis; and chronic liver disease associated with hepatitis B, C or alcohol. In another embodiment, the present disclosure provides a method for treating or preventing hyperlipidemia, where the hyperlipidemia is primary hyperlipidemia with or without a genetic component, or hyperlipidemia associated with coronary artery disease, cerebrovascular arterial disease, peripheral vascular disease, aortic aneurisms, or carotid atherosclerosis.

In one aspect, the present disclosure provides a method for treating or preventing chronic hepatitis caused by hepatitis B, C or by alcohol.

In one aspect, the present disclosure provides a method for treating or preventing other arterial disorders associated with hyperlipidemia. In one aspect, the present disclosure provides a method for treating or preventing hypertriglyceridemia.

Therapies with FXR agonists may produce various side effects, one of which is pruritus. Pruritus or itch is defined as an unpleasant sensation of the skin that provokes the urge to scratch. It is a characteristic feature of many skin diseases and an unusual sign of some systemic diseases. Pruritus may be localized or generalized and can occur as an acute or chronic condition. Itching lasting more than 6 weeks is termed chronic pruritus. Itching can be intractable and incapacitating, as well as a diagnostic and therapeutic challenge.

One of the advantages of the compositions of the present disclosure includes a decrease in the incidence and/or severity of pruritus in subjects treated with the compositions and according to the methods of the present disclosure.

In one embodiment, the incidence of pruritus decreases by at least 5%, 10%, 15%, 20%, 25%, 30%, 35%, 40%, 45%, or 50% in subjects treated with the compositions of the present disclosure. In a further embodiment, the incidence of pruritus decreases by at least 20%, 25%, 30%, 35%, 40%, 45%, or 50% in subjects treated with the compositions of the present disclosure. In a further embodiment, the incidence of pruritus decreases by at least 5%, 10%, 15%, 20%, 25%, 30%, 35%, 40%, 45%, or 50% in subjects treated with the compositions of the present disclosure during the first one month, two months, three months, four months, five months, or six months after the beginning of the treatment. In a further embodiment, the incidence of pruritus decreases by at least 20%, 25%, 30%, 35%, 40%, 45%, or 50% in subjects treated with the compositions of the present disclosure during the first one month, two months, three months, four months, five months, or six months after the beginning of the treatment.

In one embodiment, the severity of the pruritus decreases by at least 5%, 10%, 15%, 20%, 25%, 30%, 35%, 40%, 45%, or 50% in subjects treated with the compositions of the present disclosure. In a further embodiment, the severity of pruritus decreases by at least 20%, 25%, 30%, 35%, 40%, 45%, or 50% in subjects treated with the compositions of the present disclosure. In a further embodiment, the severity of pruritus decreases by at least 5%, 10%, 15%, 20%, 25%, 30%, 35%, 40%, 45%, or 50% in subjects treated with the compositions of the present disclosure during the first one month, two months, three months, four months, five months, or six months after the beginning of the treatment. In a further embodiment, the severity of pruritus decreases by at least 20%, 25%, 30%, 35%, 40%, 45%, or 50% in subjects treated with the compositions of the present disclosure during the first one month, two months, three months, four months, five months, or six months after the beginning of the treatment.

Obeticholic acid compositions described herein can be administered to a patient in an amount of between about: 1 mg to about 50 mg; 1 mg to about 40 mg; 1 mg to about 30 mg; 1 mg to about 25 mg; 1 mg to about 20 mg; 1 mg to about 10 mg; or 1 mg to about 5 mg. In one embodiment, the obeticholic acid composition can be administered to a patient in an amount of about: 5 to about 50 mg; 5 to about 40 mg; 5 to about 30 mg; 5 to about 25 mg; 5 to about 20 mg; or 5 to about 10 mg. In other instances, the obeticholic acid composition can be administered in an amount of about 1, 2, 3, 4, 5, 6, 7, 8, 9, 10, 11, 12, 13, 14, 15, 16, 17, 18, 19, 20, 21, 22, 23, 24, 25, 30, 35, 40, 45, or 50 mg. In still other instances, the obeticholic acid composition described herein can be administered at an amount of about 5 mg, 10 mg, 15 mg, 25 mg, or 50 mg. For example, an effective amount of a obeticholic acid composition described herein can be about 5 mg, 10 mg, 25 mg, or 50 mg. In another example, the amount of a starting dose of an obeticholic acid composition described herein can be about 5 mg, 10 mg, 25 mg, or 50 mg. In another example, the amount of an adjusted dose or re-adjusted dose of an obeticholic acid composition described herein can be about 5 mg, 10 mg, 25 mg, or 50 mg. It is to be understood the amount of an obeticholic acid composition described herein as administered to a patient described herein refers to the amount of obeticholic acid in the composition.

The amount of an obeticholic acid composition as provided above can refer to an effective amount as described herein. In certain embodiments, an effective amount of the obeticholic acid composition administered to a patient described herein can be 5 mg. In another embodiment, an effective amount of the obeticholic acid composition administered to a patient described herein can be 10 mg. In still another embodiment, an effective amount of the obeticholic acid composition administered to a patient described herein can be 25 mg. In yet another embodiment, an effective amount of the obeticholic acid composition administered to a patient described herein can be 50 mg.

The amount of an obeticholic acid composition as provided above can optionally refer to a starting dose administered during a titration period as described herein. In certain embodiments, a starting dose of the obeticholic acid composition administered to a patient described herein can be 5 mg. In another embodiment, a starting dose of the obeticholic acid composition administered to a patient described herein can be 10 mg. In still another embodiment, a starting dose of the obeticholic acid composition administered to a patient described herein can be 25 mg. In yet another embodiment, a starting dose of the obeticholic acid composition administered to a patient described herein can be 50 mg.

The amount of an obeticholic acid composition as provided above can refer to an adjusted dose administered after a titration period as described herein. In certain embodiments, an adjusted dose of the obeticholic acid composition administered to a patient described herein can be 5 mg. In another embodiment, an adjusted dose of the obeticholic acid composition administered to a patient described herein can be 10 mg. In still another embodiment, an adjusted dose of the obeticholic acid composition administered to a patient described herein can be 25 mg. In yet another embodiment, an adjusted dose of the obeticholic acid composition administered to a patient described herein can be 50 mg.

The amount of an obeticholic acid composition as provided above can refer to a re-adjusted dose administered after a titration period as described herein. In certain embodiments, a re-adjusted dose of the obeticholic acid composition administered to a patient described herein can be 5 mg. In another embodiment, a re-adjusted dose of the obeticholic acid composition administered to a patient described herein can be 10 mg. In still another embodiment, a re-adjusted dose of the obeticholic acid composition administered to a patient described herein can be 25 mg. In yet another embodiment, a re-adjusted dose of the obeticholic acid composition administered to a patient described herein can be 50 mg.

While it is possible to administer obeticholic acid directly without any formulation, obeticholic acid is usually administered in the form of pharmaceutical formulations comprising a pharmaceutically acceptable excipient and obeticholic acid. These formulations can be administered by a variety of routes including oral, buccal, rectal, intranasal, transdermal, subcutaneous, intravenous, intramuscular, and intranasal. Oral formulation of obeticholic acid are described further herein under the section entitled “Oral Formulation and Administration”.

In one embodiment, obeticholic acid can be administered transdermally. In order to administer transdermally, a transdermal delivery device (“patch”) is needed. Such transdermal patches may be used to provide continuous or discontinuous infusion of a compound of the present disclosure in controlled amounts. The construction and use of transdermal patches for the delivery of pharmaceutical agents is well known in the art. See, e.g., U.S. Pat. No. 5,023,252. Such patches may be constructed for continuous, pulsatile, or on demand delivery of pharmaceutical agents.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound, obeticholic acid or obeticholic acid particles, in the required amount in an appropriate solvent with one or a combination of ingredients enumerated herein, as required, followed by filtered sterilization. Generally, dispersions are prepared by incorporating the obeticholic acid into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the obeticholic acid or obeticholic acid particles, plus any additional desired ingredient from a previously sterile-filtered solution thereof.

It can be useful to orally administer an obeticholic acid composition described herein. Oral compositions generally include an inert diluent or an edible pharmaceutically acceptable carrier. They can be enclosed in gelatin capsules or compressed into tablets. For the purpose of oral therapeutic administration, the active compound, obeticholic acid or obeticholic acid particles, can be incorporated with excipients and used in the form of tablets, troches, or capsules. Oral compositions can also be prepared using a fluid carrier for use as a mouthwash, wherein the obeticholic acid or obeticholic acid particles in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as sodium starch glycolate, starch or lactose, a diluent such as microcrystalline cellulose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the obeticholic acid or obeticholic acid particles is delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the obeticholic acid or obeticholic acid particles is formulated into ointments, salves, gels, or creams as generally known in the art.

The obeticholic acid or obeticholic acid particles can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of obeticholic acid or obeticholic acid particles calculated to produce the desired therapeutic effect in association with the required pharmaceutical carrier. The specification for the dosage unit forms of the disclosure are dictated by and directly dependent on the unique characteristics of the obeticholic acid or obeticholic acid particles and the particular therapeutic effect to be achieved.

In one embodiment of the present disclosure, there is provided a pharmaceutical formulation comprising at least obeticholic acid as described above in a formulation adapted for buccal and/or sublingual, or nasal administration. This embodiment provides administration of obeticholic acid in a manner that avoids gastric complications, such as first pass metabolism by the gastric system and/or through the liver. This administration route may also reduce adsorption times, providing more rapid onset of therapeutic benefit. The compounds of the present disclosure may provide particularly favorable solubility profiles to facilitate sublingual/buccal formulations. Such formulations typically require relatively high concentrations of active ingredients to deliver sufficient amounts of active ingredients to the limited surface area of the sublingual/buccal mucosa for the relatively short durations the formulation is in contact with the surface area, to allow the absorption of the active ingredient. Thus, the very high activity of obeticholic acid, combined with its high solubility, facilitates its suitability for sublingual/buccal formulation.

Obeticholic acid is preferably formulated in a unit dosage form, each dosage containing from about 0.05 mg to about 1500 mg. In another embodiment, the formulation comprises about 0.05 mg to about 100 mg. In yet another embodiment, the formulation comprises about 1 mg to about 100 mg. In another embodiment, the formulation comprises about 0.05 mg to about 50 mg. In yet another embodiment, the formulation comprises about 0.05 mg to about 30 mg. In another embodiment, the formulation comprises about 0.05 mg to about 20 mg. In yet another embodiment, the formulation comprises about 0.5 mg to about 30 mg. In another embodiment, the formulation comprises about 0.5 mg to about 25 mg. In yet another embodiment, the formulation comprises about 1 mg to about 25 mg. In another embodiment, the formulation comprises about 4 mg to about 26 mg. In another embodiment, the formulation comprises about 5 mg to about 25 mg. In yet another embodiment, the formulation comprises about 0.05 mg to about 2 mg. In another embodiment, the formulation comprises about 1 mg to about 2 mg. In one embodiment, the formulation comprises about 1.2 mg to about 1.8 mg. In one embodiment, the formulation comprises about 1.3 mg to about 1.7 mg. In one embodiment, the formulation comprises about 1.5 mg. In one embodiment, the formulation comprises about 0.05 mg to about 0.5 mg. In another embodiment, the formulation comprises about 0.08 mg to about 0.8 mg. In yet another embodiment, the formulation comprises about 0.1 mg to about 0.5 mg. In another embodiment, the formulation comprises about 0.25 mg.

Obeticholic acid is generally effective over a wide dosage range. For examples, dosages per day normally fall within the range of about 0.0001 to about 30 mg/kg of body weight. In the treatment of adult humans, the range of about 0.1 to about 15 mg/kg/day, in single or divided dose, is especially preferred. In one embodiment, the formulation comprises about 0.05 mg to about 1500 mg. In another embodiment, the formulation comprises about 0.05 mg to about 100 mg. In yet another embodiment, the formulation comprises about 1 mg to about 100 mg. In another embodiment, the formulation comprises about 0.05 mg to about 50 mg. In another embodiment, the formulation comprises about 0.05 mg to about 30 mg. In yet another embodiment, the formulation comprises about 0.05 mg to about 20 mg. In yet another embodiment, the formulation comprises about 0.05 mg to about 10 mg.

In one embodiment, the formulation comprises about 3 mg to about 30 mg. In another embodiment, the formulation comprises about 0.05 mg to about 25 mg. In another embodiment, the formulation comprises about 4 mg to about 25 mg. In another embodiment, the formulation comprises about 5 mg to about 25 mg. In another embodiment, the formulation comprises about 5 mg to about 10 mg. In one embodiment, the formulation comprises about 1 mg to about 2 mg. In one embodiment, the formulation comprises about 1.2 mg to about 1.8 mg. In one embodiment, the formulation comprises about 1.3 mg to about 1.7 mg. In one embodiment, the formulation comprises about 0.05 mg to about 0.5 mg. In another embodiment, the formulation comprises about 0.08 mg to about 0.8 mg. In yet another embodiment, the formulation comprises about 0.1 mg to about 0.5 mg. In another embodiment, the formulation comprises about 25 mg. In another embodiment, the formulation comprises about 10 mg. In one embodiment, the formulation comprises about 5 mg. In another embodiment, the formulation comprises about 1 mg. However, it will be understood that the amount of obeticholic acid actually administered will be determined by a physician, in the light of the relevant circumstances, including the condition to be treated, the chosen route of administration, the form of obeticholic acid administered, the age, weight, and response of the individual patient, and the severity of the patient's symptoms, and therefore the above dosage ranges are not intended to limit the scope of the disclosure in any way. In some instances, dosage levels below the lower limit of the aforesaid range may be more than adequate, while in other cases still larger doses may be employed without causing any harmful side effect, provided that such larger doses are first divided into several smaller doses for administration throughout the day.

Obeticholic acid compositions described herein can be administered in accordance with a dosing regimen. A dosing regimen refers to continual and intermittent administration of a obeticholic acid composition described herein at one or more of the amounts described herein. Thus, in certain instances, a dosing regimen can include administration of a obeticholic acid composition described herein continually for any number of days, weeks, months, or years as set forth herein. In other instances, a dosing regimen can include administration of a obeticholic acid composition described herein intermittently, where, for example, the composition is administered for one period of time followed by a rest period or off period where the obeticholic acid composition is not administered.

Obeticholic acid compositions useful in the methods of treating described herein include administration of such compositions daily (QD), every other day (Q2D), once a week (QW), twice a week (BID), three times a week (TIW), once a month (QM), or twice a month (Q2M). In one embodiment, a obeticholic acid composition described herein is administered QD. Thus, an effective amount of an obeticholic acid composition described herein can be administered QD to treat a disease or condition described herein. A starting dose described herein can be administered QD during the course of a titration period described herein to treat a disease or condition described herein. An adjusted dose described herein can be administered QD to treat a disease or condition described herein.

In another embodiment, an obeticholic acid composition described herein is administered Q2D. An effective amount of an obeticholic acid composition described herein can be administered Q2D to treat a disease or condition described herein. A starting dose described herein can be administered Q2D during the course of a titration period described herein to treat a disease or condition described herein. An adjusted dose described herein can be administered Q2D to treat a disease or condition described herein.

In another embodiment, an obeticholic acid composition is described herein administered QW. An effective amount of an obeticholic acid composition described herein can be administered QW to treat a disease or condition described herein. A starting dose described herein can be administered QW during the course of a titration period described herein to treat a disease or condition described herein. An adjusted dose described herein can be administered QW to treat a disease or condition described herein.

In another embodiment, an obeticholic acid composition is described herein administered BID. An effective amount of an obeticholic acid composition described herein can be administered BID to treat a disease or condition described herein. A starting dose described herein can be administered BID during the course of a titration period described herein to treat a disease or condition described herein. An adjusted dose described herein can be administered BID to treat a disease or condition described herein.

In another embodiment, an obeticholic acid composition is described herein administered TIW. An effective amount of an obeticholic acid composition described herein can be administered TIW to treat a disease or condition described herein. A starting dose described herein can be administered TIW during the course of a titration period described herein to treat a disease or condition described herein. An adjusted dose described herein can be administered TIW to treat a disease or condition described herein.

In another embodiment, an obeticholic acid composition is described herein administered QM. An effective amount of an obeticholic acid composition described herein can be administered QM to treat a disease or condition described herein. A starting dose described herein can be administered QM during the course of a titration period described herein to treat a disease or condition described herein. An adjusted dose described herein can be administered QM to treat a disease or condition described herein.

In another embodiment, an obeticholic acid composition is described herein administered Q2M. An effective amount of an obeticholic acid composition described herein can be administered Q2M to treat a disease or condition described herein. A starting dose described herein can be administered Q2M during the course of a titration period described herein to treat a disease or condition described herein. An adjusted dose described herein can be administered Q2M to treat a disease or condition described herein.

The embodiments described above include administration at an amount described above. For example, an obeticholic acid composition described herein can be administered in a frequency provided above in an amount of 5 mg, 10 mg, 25 mg, or 50 mg.

Dosing regimens of the obeticholic acid compositions described herein useful for treating diseases and conditions described herein can include a titration period. A titration period typically includes a lower dosage of an obeticholic acid composition described herein for a period of time. In certain instances, and without being bound by any particular theory, administration using a titration period described herein can decrease or eliminate the onset of adverse effects. In other instances, and without being bound by any particular theory, administration using a titration period described herein can permit increased dosages of obeticholic acid compositions described herein to an individual over the course of a treatment.

A titration period can be a period of time of about: 1 month to about 24 months; 1 month to about 21 months; 1 month to about 18 months; 1 month to about 15 months; 1 month to about 12 months; 1 month to about 9 months; 1 month to about 6 months; or 1 month to about 3 months. In another embodiment, a titration period includes a time of about: 3 months to about 24 months; 3 months to about 21 months; 3 months to about 18 months; 3 months to about 15 months; 3 months to about 12 months; 3 or months to about 6 months. In still another embodiment, a titration period includes a time of about: 6 months to about 24 months; 6 months to about 21 months; 6 months to about 18 months; 6 months to about 15 months; or 6 months to about 12 months. In yet another embodiment, a titration period includes a time of about: 2 months to about 4 months; 2 months to about 7 months; 2 months to about 8 months; 4 months to about 8 months; 5 months to about 7 months; or 5 months to about 8 months. For example, a titration period can be about 1 to about 6 months. In another example, a titration period can be about 3 to about 6 months.

A titration period can include a time of about 1, 2, 3, 4, 5, 6, 7, 8, 9, 10, 11, 12, 13, 14, 15, 16, 17, 18, 19, 20, 21, 22, 23, or 24 months. In certain embodiments, a titration period includes a time of about 1, 2, 3, 4, 5, 6, 7, 8, 9, 10, 11, or 12 months. In another embodiment, the titration period includes a time of about 1, 2, 3, 4, 5, 6, 7, 8, or 9 months. In another embodiment, the titration period includes a time of about 1, 2, 3, 4, 5, or 6 months. In another embodiment, the titration period includes a time of about 1, 2, or 3 months. For example, a titration period can be about 1 month. In another example a titration period can be about 2 months. In another example a titration period can be about 3 months. In still another example a titration period can be about 4 months. In yet another example a titration period can be about 5 months. In another example a titration period can be about 6 months. In one example a titration period is 3 months or 6 months. In another example a titration period can be about 7 months. In another example a titration period can be about 8 months. In another example a titration period can be about 9 months.

As provided above, the amounts of a obeticholic acid composition described herein, optionally administered in a titration period can be reduced compared to an adjusted amount as described herein. Accordingly, provided herein are treatment regimens that include administering obeticholic acid compositions described herein for the treatment of a disease or condition described herein (e.g., NASH or compensated cirrhosis) wherein the starting dose administered during a titration period described above is lower than the amount of an adjusted dose administered after a titration period. Still further provided herein are treatment regimens that include administering obeticholic acid compositions described herein for the treatment of a disease or condition described herein (e.g., NASH or compensated cirrhosis) where the starting dose administered during a titration period described above is lower than the amount of an adjusted dose administered after a titration period and where the frequency of administration (e.g., QD, Q2D, or QW) for the adjusted dose is greater than the frequency of administration of the starting dose. Still further provided herein are treatment regimens that include administering obeticholic acid compositions described herein for the treatment of a disease or condition described herein (e.g., NASH with fibrosis or compensated cirrhosis due to NASH) where the starting dose administered during a titration period described above is lower than the amount of an adjusted dose administered after a titration period and where the frequency of administration (e.g., QD, Q2D, or QW) for the adjusted dose is less than the frequency of administration of the starting dose. Increases in the adjusted dose (or any re-adjusted dose) can be performed after the patient's liver function is assessed, monitored, or measured as described herein, where the liver function is considered not-impaired.

In embodiments, the adjusted dose can be increased compared to the starting dose when the level of ALP is about equal to or is not reduced compared to a control as described herein. In embodiments, the adjusted can be increased compared to the starting dose when the level of bilirubin is about equal to or is not reduced compared to a control as described herein. In embodiments, the adjusted dose can be increased compared to the starting dose when the level of ALP and bilirubin are about equal to or are not reduced compared to a control as described herein. In certain instances, the adjusted dose can be increased compared to the starting dose where a patient described herein tolerates the starting dose amount. In certain embodiments, the starting dose can be 5 mg. In certain embodiments, the starting dose is 10 mg. In certain embodiments, the starting dose is 5 mg and the adjusted dose is greater than 5 mg (e.g., about 6 mg to about 50 mg). In one embodiment, the starting dose is 5 mg and the adjusted dose is 10 mg. In one embodiment, the starting dose is 10 mg and the adjusted dose is 25 mg.

In embodiments, the adjusted dose can be increased compared to the starting dose when a measured DSI score (measured using a HepQuant® test, such as a HepQuant®-SHUNT test) is about equal to or is not reduced compared to a control as described herein. In certain instances, the adjusted dose can be increased compared to the starting dose where a patient described herein tolerates the starting dose amount. In certain embodiments, the starting dose can be 5 mg. In certain embodiments, the starting dose is 10 mg. In certain embodiments, the starting dose is 5 mg and the adjusted dose is greater than 5 mg (e.g., about 6 mg to about 50 mg). In one embodiment, the starting dose is 5 mg and the adjusted dose is 10 mg. In one embodiment, the starting dose is 10 mg and the adjusted dose is 25 mg.

Also provided herein are treatment regimens that include administering obeticholic acid compositions described herein for the treatment of a disease or condition described herein (e.g., NASH with fibrosis or compensated cirrhosis due to NASH) where the starting dose administered during a titration period described above is equal to the amount of an adjusted dose administered after a titration period. Further provided herein are treatment regimens that include administering obeticholic acid compositions described herein for the treatment of a disease or condition described herein (e.g., NASH with fibrosis or compensated cirrhosis due to NASH) where the starting dose administered during a titration period described above is equal to the amount of an adjusted dose administered after a titration period and where the frequency of administration (e.g., QD, Q2D, or QW) for the starting dose is the same as the adjusted dose. Still further provided herein are treatment regimens that include administering obeticholic acid compositions described herein for the treatment of a disease or condition described herein (e.g. NASH or compensated cirrhosis) where the starting dose administered during a titration period described above is equal to the amount of an adjusted dose administered after a titration period and where the frequency of administration (e.g., QD, Q2D, or QW) for the adjusted dose is greater than the frequency of administration of the starting dose. Still further provided herein are treatment regimens that include administering obeticholic acid compositions described herein for the treatment of a disease or condition described herein (e.g., NASH or compensated cirrhosis) where the starting dose administered during a titration period described above is equal to the amount of an adjusted dose administered after a titration period and where the frequency of administration (e.g., QD, Q2D, or QW) for the adjusted dose is less than the frequency of administration of the starting dose. The adjusted dose (or any re-adjusted dose) can be equal to the starting dose where the patient's liver function is assessed, monitored, or measured as described herein, where the liver function is considered not-impaired.

In embodiments, the adjusted dose can be equal to the starting dose when the level of ALP is reduced compared to a control as described herein. In embodiments, the adjusted dose can be equal to the starting dose when the level of bilirubin is reduced compared to a control as described herein. In embodiments, the adjusted dose can be equal to the starting dose when the level of ALP and bilirubin are reduced compared to a control as described herein. In certain instances, the adjusted dose can be equal to the starting dose where a patient described herein tolerates or poorly tolerates (e.g., has onset of adverse effects described herein) the starting dose amount. In certain embodiments, the starting dose can be 5 mg. In certain embodiments, the starting dose is 10 mg. In certain embodiments, the starting dose is 5 mg and the adjusted dose is 5 mg. In one embodiment, the starting dose is 10 mg and the adjusted dose is 10 mg. A starting does can be 10 mg and the adjusted dose can be 25 mg.

In embodiments, the adjusted dose can be equal to the starting dose when a measured DSI score (measured using a HepQuant® test, such as a HepQuant®-SHUNT test) is reduced compared to a control as described herein. In certain instances, the adjusted dose can be equal to the starting dose where a patient described herein tolerates or poorly tolerates (e.g., has onset of adverse effects described herein) the starting dose amount. In certain embodiments, the starting dose can be 5 mg. In certain embodiments, the starting dose is 10 mg. In certain embodiments, the starting dose is 5 mg and the adjusted dose is 5 mg. In one embodiment, the starting dose is 10 mg and the adjusted dose is 10 mg.

Further provided herein are treatment regimens that optionally include a starting dose and an adjusted dose as provided in the regimens above, where the adjusted dose is further reduced during the course of treatment. In certain instances, the adjusted dose is reduced to a new re-adjusted dose having a decreased amount of an obeticholic acid composition described herein. In other instances the adjusted dose is reduced to a new re-adjusted dose having the same amount of an obeticholic acid composition described herein but a decreased frequency of administration (e.g., from QD to Q2D or QW). In still other instances, the adjusted dose is modified such that the re-adjusted dose includes a decreased amount of an obeticholic acid composition described herein and is administered at a decreased frequency compared to the adjusted dose.

Dosing regimens of the obeticholic acid compositions described herein useful for treating diseases and conditions described herein can include initial doses and at least one adjusted dose. An initial dose is the amount of obeticholic acid this is initially given to the subject. This initial dose can be continued to be administered to the subject over the course of an initial period.

An initial period can be a period of time of about: 1 month to about 24 months; 1 month to about 21 months; 1 month to about 18 months; 1 month to about 15 months; 1 month to about 12 months; 1 month to about 9 months; 1 month to about 6 months; or 1 month to about 3 months. In another embodiment, a initial period includes a time of about: 3 months to about 24 months; 3 months to about 21 months; 3 months to about 18 months; 3 months to about 15 months; 3 months to about 12 months; 3 or months to about 6 months. In still another embodiment, a initial period includes a time of about: 6 months to about 24 months; 6 months to about 21 months; 6 months to about 18 months; 6 months to about 15 months; or 6 months to about 12 months. In yet another embodiment, a initial period includes a time of about: 2 months to about 4 months; 2 months to about 7 months; 2 months to about 8 months; 4 months to about 8 months; 5 months to about 7 months; or 5 months to about 8 months. For example, a initial period can be about 1 to about 6 months. In another example, a initial period can be about 3 to about 6 months.

A initial period can include a time of about 1, 2, 3, 4, 5, 6, 7, 8, 9, 10, 11, 12, 13, 14, 15, 16, 17, 18, 19, 20, 21, 22, 23, or 24 months. In certain embodiments, a initial period includes a time of about 1, 2, 3, 4, 5, 6, 7, 8, 9, 10, 11, or 12 months. In another embodiment, the initial period includes a time of about 1, 2, 3, 4, 5, 6, 7, 8, or 9 months. In another embodiment, the initial period includes a time of about 1, 2, 3, 4, 5, or 6 months. In another embodiment, the initial period includes a time of about 1, 2, or 3 months. For example, a initial period can be about 1 month. In another example a initial period can be about 2 months. In another example a initial period can be about 3 months. In still another example a initial period can be about 4 months. In yet another example a initial period can be about 5 months. In another example a initial period can be about 6 months. In one example a initial period is 3 months or 6 months. In another example a initial period can be about 7 months. In another example a initial period can be about 8 months. In another example a initial period can be about 9 months.

At any point of time during an initial period or alternatively at the end of an initial period, the subject's liver function can be measured, using, for example a HebQuant® test (e.g. a HebQuant®-SHUNT test). The results of the liver function test can be used to determine an adjusted dose, i.e. a dose that is different than the initial dose. In one non-limiting example, if the test of the subject's liver function reveals that the initial dose has not been effective, the adjusted dose can be greater than the initial dose. In another non-limiting example, if the test of the subject's liver function reveals an improvement in liver function with the initial dose, the adjusted dose may be less than the initial dose as the patient requires less therapeutic intervention.

As provided above, the amounts of a obeticholic acid composition described herein, optionally administered in an initial period (the initial dose) can be reduced compared to an adjusted amount (adjusted dose) as described herein. Accordingly, provided herein are treatment regimens that include administering obeticholic acid compositions described herein for the treatment of a disease or condition described herein (e.g., NASH with fibrosis or compensated cirrhosis due to NASH) wherein the initial dose administered during an initial period described above is lower than the amount of an adjusted dose administered after the initial period. Still further provided herein are treatment regimens that include administering obeticholic acid compositions described herein for the treatment of a disease or condition described herein (e.g., NASH or compensated cirrhosis) where the initial dose administered during an initial period described above is lower than the amount of an adjusted dose administered after the initial period and where the frequency of administration (e.g., QD, Q2D, or QW) for the adjusted dose is greater than the frequency of administration of the initial dose. Still further provided herein are treatment regimens that include administering obeticholic acid compositions described herein for the treatment of a disease or condition described herein (e.g., NASH with fibrosis or compensated cirrhosis due to NASH) where the initial dose administered during an initial period described above is lower than the amount of an adjusted dose administered after the initial period and where the frequency of administration (e.g., QD, Q2D, or QW) for the adjusted dose is less than the frequency of administration of the initial dose. Increases in the adjusted dose (or any re-adjusted dose) can be performed after the patient's liver function is assessed, monitored, or measured as described herein, where the liver function is considered not-impaired.

In embodiments, the adjusted dose can be increased compared to the starting dose when the level of ALP is about equal to or is not reduced compared to a control as described herein. In embodiments, the adjusted can be increased compared to the starting dose when the level of bilirubin is about equal to or is not reduced compared to a control as described herein. In embodiments, the adjusted dose can be increased compared to the starting dose when the level of ALP and bilirubin are about equal to or are not reduced compared to a control as described herein. In certain instances, the adjusted dose can be increased compared to the starting dose where a patient described herein tolerates the starting dose amount. In certain embodiments, the starting dose can be 5 mg. In certain embodiments, the starting dose is 10 mg. In certain embodiments, the starting dose is 5 mg and the adjusted dose is greater than 5 mg (e.g., about 6 mg to about 50 mg). In one embodiment, the starting dose is 5 mg and the adjusted dose is 10 mg. In embodiments, the initial dose is 10 mg and the adjusted dose is 25 mg.

In embodiments, the adjusted dose can be increased compared to the starting dose when a measured DSI score (measured using a HepQuant® test, such as a HepQuant®-SHUNT test) is about equal to or is not reduced compared to a control as described herein. In certain instances, the adjusted dose can be increased compared to the starting dose where a patient described herein tolerates the starting dose amount. In certain embodiments, the starting dose can be 5 mg. In certain embodiments, the starting dose is 10 mg. In certain embodiments, the starting dose is 5 mg and the adjusted dose is greater than 5 mg (e.g., about 6 mg to about 50 mg). In one embodiment, the starting dose is 5 mg and the adjusted dose is 10 mg. In one embodiment, the starting dose is 10 mg and the adjusted dose is 25 mg.

Also provided herein are treatment regimens that include administering obeticholic acid compositions described herein for the treatment of a disease or condition described herein (e.g., NASH with fibrosis or compensated cirrhosis due to NASH) where the initial dose administered during a titration period described above is equal to the amount of an adjusted dose administered after a titration period. Further provided herein are treatment regimens that include administering obeticholic acid compositions described herein for the treatment of a disease or condition described herein (e.g., NASH with fibrosis or compensated cirrhosis due to NASH) where the initial dose administered during a titration period described above is equal to the amount of an adjusted dose administered after a titration period and where the frequency of administration (e.g., QD, Q2D, or QW) for the initial dose is the same as the adjusted dose. Still further provided herein are treatment regimens that include administering obeticholic acid compositions described herein for the treatment of a disease or condition described herein (e.g. NASH or compensated cirrhosis) where the initial dose administered during a titration period described above is equal to the amount of an adjusted dose administered after a titration period and where the frequency of administration (e.g., QD, Q2D, or QW) for the adjusted dose is greater than the frequency of administration of the initial dose. Still further provided herein are treatment regimens that include administering obeticholic acid compositions described herein for the treatment of a disease or condition described herein (e.g., NASH or compensated cirrhosis) where the initial dose administered during a titration period described above is equal to the amount of an adjusted dose administered after a titration period and where the frequency of administration (e.g., QD, Q2D, or QW) for the adjusted dose is less than the frequency of administration of the initial dose. The adjusted dose (or any re-adjusted dose) can be equal to the initial dose where the patient's liver function is assessed, monitored, or measured as described herein, where the liver function is considered not-impaired.

In embodiments, the adjusted dose can be equal to the starting dose when the level of ALP is reduced compared to a control as described herein. In embodiments, the adjusted dose can be equal to the starting dose when the level of bilirubin is reduced compared to a control as described herein. In embodiments, the adjusted dose can be equal to the starting dose when the level of ALP and bilirubin are reduced compared to a control as described herein. In certain instances, the adjusted dose can be equal to the starting dose where a patient described herein tolerates or poorly tolerates (e.g., has onset of adverse effects described herein) the starting dose amount. In certain embodiments, the starting dose can be 5 mg. In certain embodiments, the starting dose is 10 mg. In certain embodiments, the starting dose is 5 mg and the adjusted dose is 5 mg. In one embodiment, the starting dose is 10 mg and the adjusted dose is 10 mg. In one embodiment, the initial dose is 25 mg and the adjusted dose is 25 mg.

In embodiments, the adjusted dose can be equal to the starting dose when a measured DSI score (measured using a HepQuant® test, such as a HepQuant®-SHUNT test) is reduced compared to a control as described herein. In certain instances, the adjusted dose can be equal to the starting dose where a patient described herein tolerates or poorly tolerates (e.g., has onset of adverse effects described herein) the starting dose amount. In certain embodiments, the starting dose can be 5 mg. In certain embodiments, the starting dose is 10 mg. In certain embodiments, the starting dose is 5 mg and the adjusted dose is 5 mg. In one embodiment, the starting dose is 10 mg and the adjusted dose is 10 mg.

Further provided herein are treatment regimens that optionally include an initial dose and an adjusted dose as provided in the regimens above, where the adjusted dose is further reduced during the course of treatment. In certain instances, the adjusted dose is reduced to a new re-adjusted dose having a decreased amount of an obeticholic acid composition described herein. In other instances the adjusted dose is reduced to a new re-adjusted dose having the same amount of an obeticholic acid composition described herein but a decreased frequency of administration (e.g., from QD to Q2D or QW). In still other instances, the adjusted dose is modified such that the re-adjusted dose includes a decreased amount of an obeticholic acid composition described herein and is administered at a decreased frequency compared to the adjusted dose.

The obeticholic acid composition described herein can be administered for any number of days, weeks, months, or years, including indefinitely, provided that the dosage remains efficacious for the patient and the patient tolerates the dosage (e.g., an adjusted or re-adjusted dose as described herein). In certain instances, an obeticholic acid composition described herein is administered to a patient described herein until loss of efficacy, or until development of unacceptable toxicity or undesired adverse effects, such as, for example, those described herein. Daily dosing of an obeticholic acid composition described herein can be dependent upon patient tolerance to the dosage, composition, or frequency of administration. For example, daily dosing can be administered to a patient described herein where the patient tolerates a daily dosage amount (e.g., 5 mg, 10 mg, 25 mg, or 50 mg). Alternatively or additionally, the daily dosing can be modified to increase or reduce the amount of an obeticholic acid composition described herein as provided above where the patient is tolerant or is intolerant to the dose, respectively. In certain embodiments, modification of the adjusted dose (or any re-adjusted dose) can be performed after the patient's liver function is assessed, monitored, or measured as described herein. In certain instances, the adjusted dose (or re-adjusted dose) is increased or maintained (e.g., equivalent to a starting dose) where the liver function is not-impaired. In other instances, the adjusted dose (or re-adjusted dose) is decreased or maintained (e.g., equivalent to a starting dose) where the patient's liver function is impaired.

The amount of an obeticholic acid described herein administered to a patient described herein can be modified as a result of intolerability or development of one or more adverse effects such as those described herein. For example, in one instance the amount of an obeticholic acid composition described herein administered to a patient can be changed from a QD dosage to a Q2D dosage. In certain embodiments, the dosage of an obeticholic acid described herein is modified from a QD to Q2D dosage upon development of an adverse effect described herein (e.g., severe pruritus). In one example, administration of an obeticholic acid composition described herein at 5 mg QD can be modified to a 5 mg Q2D dosage. Such a modification can reduce or eliminate undesired adverse effects while maintaining the desired efficacy. In another example, administration of an obeticholic acid composition described herein at 10 mg QD can be reduced to 5 mg QD. It should be understood that exemplary dosing regimens described herein can be combined. For example, a reduced dosage of an obeticholic acid composition described herein from 10 mg to 5 mg QD could be further reduced to a 5 mg Q2D dosage where undesired adverse effects remain. In still another example, dosing of the obeticholic acid composition can be temporarily suspended (e.g., an off period) for about 1, 2, 3, 4, 5, 6, 7, 8, 9, 10, 11, 12, 13, or 14 days, or 1, 2, 3, or 4 weeks.

In one exemplary dosing regimen, a NASH patient (e.g., NASH patient with fibrosis or a patient with compensated cirrhosis due to NASH) is administered an obeticholic acid composition described herein where: the starting dose of the obeticholic acid composition described herein is administered QD to a patient described herein at an amount of 5 mg and the obeticholic acid composition is administered QD to the patient in an adjusted dose of 5 mg. The exemplary dosing regimen can include a titration period of about 1 to about 6 months.

In another exemplary dosing regimen, a NASH patient (e.g., NASH patient with fibrosis or a patient with compensated cirrhosis due to NASH) is administered an obeticholic acid composition described herein where: the starting dose of the obeticholic acid composition described herein is administered QD to a patient described herein at an amount of 5 mg in a titration period of about 3 months or about 6 months and the obeticholic acid composition is administered QD to the patient in an adjusted dose of 5 mg.

In still another exemplary dosing regimen, a NASH patient (e.g., NASH patient with fibrosis or a patient with compensated cirrhosis due to NASH) is administered an obeticholic acid composition described herein where: the starting dose of the obeticholic acid composition described herein is administered QD to a patient in a titration period of about 3 months or about 6 months and the obeticholic acid composition is administered QD to the patient in an adjusted dose of 10 mg.

In another exemplary dosing regimen, a NASH patient (e.g., NASH patient with fibrosis or a patient with compensated cirrhosis due to NASH) is administered an obeticholic acid composition described herein where: the starting dose of the obeticholic acid composition described herein is administered QD to a patient in a titration period of about 3 months or about 6 months and the obeticholic acid composition is administered QD to the patient in an adjusted dose of 5 mg, where the adjusted dose is modified to a 5 mg Q2D re-adjusted dose upon development of an adverse effect (e.g., pruritus or severe pruritus).

In still another exemplary dosing regimen, a NASH patient (e.g., NASH patient with fibrosis or a patient with compensated cirrhosis due to NASH) is administered a obeticholic acid composition described herein, where the starting dose of the obeticholic acid composition described herein is administered QD to a patient in a titration period of about 3 months or about 6 months and the obeticholic acid composition is administered QD to the patient in an adjusted dose of 10 mg, where the adjusted dose is subsequently modified to a 5 mg QD re-adjusted dose upon development of an adverse effect (e.g., pruritus or severe pruritus).

The amount of an obeticholic acid composition described herein administered to a patient can be determined by the existence of any preexisting conditions in the patient. For example, where a patient described herein has or has had hepatic impairment, the dosage of the obeticholic acid composition described herein can be modified. In certain instances, the hepatic impairment is a Child-Pugh Class A, Class B or Class C hepatic impairment. In one embodiment, the hepatic impairment is Child-Pugh Class A. In one embodiment, the hepatic impairment is Child-Pugh Class B. In one embodiment, the hepatic impairment is Child-Pugh Class C. In such instances, the amount of an obeticholic acid composition described herein can be administered in a decreased amount during and after a titration period when compared to administration of the same obeticholic acid composition to a patient who does not have hepatic impairment.

In one example dosing regimen, a patient having hepatic impairment is administered an obeticholic acid composition described herein at an amount of about 1 mg to about 5 mg, where the composition is administered at least once weekly (QW). In one instance, the obeticholic acid composition described herein is administered at an amount of about 5 mg once weekly to a patient diagnosed with hepatic impairment (e.g., Child-Pugh Class B or C).

For example, the dosing regimen can include administering an obeticholic acid composition described herein to a patient having hepatic impairment, where the obeticholic acid composition is administered at a starting dose of 5 mg QW for a titration period of 3 or 6 months and administered at an adjusted dose of 5 mg QW. The patient's liver function can be assessed, monitored, or measured as described herein. Where the patient's liver function is not impaired, the adjusted dose can be increased to a re-adjusted dose of 5 mg administered BIW or 5 mg QD.

In certain instances, a patient can develop liver impairment during the course of administration. It is understood, using the disclosure provided herein, that the adjusted dose can be decreased in amount or frequency to avoid progression of liver impairment.

Further provided herein is a method of treating NASH in a patient in need thereof by administering an effective amount of a obeticholic acid composition described herein QD, where the effective amount is either a 5 mg or 10 mg dose. In another aspect is a method of treating NASH in a patient in need thereof by administering a starting dose of 5 mg QD of an obeticholic acid composition described herein for at least 3 months; evaluating the tolerance of the patient, the patient's liver function as described herein, and/or the efficacy of treatment, where patient tolerance, liver function, and/or lowered efficacy indicate end of a titration period and administration of an adjusted dose of 10 mg QD. In one embodiment, the patient tolerance, liver function, and/or lowered efficacy indicate end of a titration period and administration of an adjusted dose of 5 mg QD.

In embodiments described herein, an obeticholic acid composition described herein can be metabolized to a obeticholic acid conjugate, such as for example, a glycine, taurine, or sarcosine conjugate of obeticholic acid. Such metabolites can be useful in treating a disease or condition provided herein. In certain instances, production of conjugates can be assessed, monitored, measured, or detected, as described herein during the course of a treatment. In some embodiments, increased levels of obeticholic acid conjugates can result in adjusted dosages of an obeticholic acid composition described herein.

In another example, the active agent is a peroxisome proliferator-activated receptor alpha (PPARα) agonist, a peroxisome proliferator-activated receptor delta (PPARδ) agonist, a dual PPARα/δ agonist, a dual PPARα/γ agonist, or pan-PPAR agonist, an HMG CoA reductase inhibitor, a GLP1 agonist, insulin, insulin mimetic, metformin, a GTP4 agonist, an HST2 inhibitor, a DPP-IV inhibitor, an SGLT2 inhibitor or a hydroxysteroid dehydrogenase (HSD) inhibitor, such as an 11β-HSD1 inhibitor, an ASK1 inhibitor, an ACC1 inhibitor, a NOX1 and/or NOX4 inhibitor, an inhibitor or antagonist of one or more chemokine receptors, such as, for example, CCR2 and CCR5.

Patients described herein include patients having a disease or condition described herein. A patient can be described or referred to by the condition treated. For example, a patient having NASH can be referred to herein as a NASH patient. A patient described herein can have a preexisting condition (e.g., a condition other than the disease or condition treated by the obeticholic acid composition described herein that existed at the time of first administration). In one instance a patient described herein has hepatic impairment. In another instance a patient described herein has renal impairment. In yet another instance the patient is an elderly/geriatric patient. In another instance the patient is a pediatric patient.

In some embodiments, administration of an obeticholic acid composition described herein together with certain contra-active agents can result in (1) decreased efficacy of the obeticholic acid composition and/or (2) development of toxicity or adverse effects described herein. For example, administration of an obeticholic acid composition described herein with blood clotting and anti-coagulation agents can result in decreased International Normalized Ratio (INR). In certain instances, coagulation and anti-coagulation agents can be administered in combination with an obeticholic acid composition described herein by monitoring fluctuations of the INR of the patient and adjusting dosages as understood in the art to maintain proper INR.

In one embodiment, the compositions described herein reduce adverse effects associated with other formulations (e.g., larger particle sized obeticholic acid). For example, an obeticholic acid composition described herein when administered to a patient described herein for a condition or disease described herein can reduce one or more adverse effects selected from Hepatic encephalopathy, ascites, variceal bleeding, skin eruptions, prurigo, pruritus (including generalized, eye, anal, vulvovaginal and rash), fatigue, asthenia, abdominal pain (including upper and lower pain and tenderness), abdominal discomfort, gastrointestinal pain, dizziness, urticaria (including cholinergic), rashes (including macular, popular, maculo-papular, and heat rashes), arthralgia, oropharyngeal pain, cough, constipation, edemal peripheral, palpitations, pyrexia, eczema, and procedural pain. In certain instances, the one or more adverse effects that are reduced include pruritus. It was discovered, inter alia, that titration of an obeticholic acid composition described herein can reduce the incidence of or mean time until onset of severe pruritus.

In another embodiment, the obeticholic acid compositions described herein include reduced levels of impurities commonly found in the synthesis of obeticholic acid. 6α-ethylursodeoxycholic acid (6-EUDCA), 3α-hydroxy-6α-ethyl-7-keto-5β-cholan-24-oic acid, 6β-ethylchenodeoxycholic acid; 3α,7α-dihydroxy-6β-ethyl-5β-cholan-24-oic acid, 3α,7α-dihydroxy-6-ethyliden-5β-cholan-24-oic acid, Chenodeoxycholic acid (CDCA); 3α,7α-dihydroxy-5β-cholan-24-oic acid, dimer of OCA, 3α-(3α,7α-dihydroxy-6α-ethyl-5β-cholan-24-oyloxy)-7α-hydroxy-6α-ethyl-5β-cholan-24-oic acid, or 3α-O-Acetyl-6α-ethylchenodeoxycholic acid; 3α-O-acetyl-7α-hydroxy-6α-ethyl-5β-cholan-24-oic acid.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The disclosure having now been described by way of written description, those of skill in the art will recognize that the disclosure can be practiced in a variety of embodiments and that the foregoing description and examples below are for purposes of illustration and not limitation of the claims that follow.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended. It is to be further understood that resort may be had to various other embodiments, modifications, and equivalents thereof which may suggest themselves to those skilled in the art without departing from the spirit of the present disclosure and/or scope of the appended claims.

### Example 1: A Phase 1, Double-Blind, Randomized, Placebo-Controlled Study to Evaluate Safety, Pharmacokinetics, and Pharmacodynamics of Obeticholic Acid in Subjects with Nonalcoholic Steatohepatitis with Fibrosis

The primary objectives of this study are to evaluate the safety and tolerability of OCA treatment in subjects with NASH with fibrosis and to evaluate single- and multiple-dose pharmacokinetics of OCA, its conjugates, and metabolites in subjects with NASH with fibrosis.

The secondary objectives of this study are to evaluate the pharmacodynamics of OCA on FXR activation and to evaluate liver chemistries and the pharmacodynamics of OCA on biomarkers of NASH, including inflammation, apoptosis, and oxidation stress.

Additional objectives of this study include evaluating the pharmacodynamics of OCA on blood biomarkers of fibrosis, evaluating the effect of OCA on anthropometric measures, evaluating the effect of OCA on the subject's micro ribonucleic acid (miRNA) profile, evaluating the effect of OCA on glycemic control and lipid markers and evaluating the pharmacokinetic and pharmacodynamic relationships of OCA.

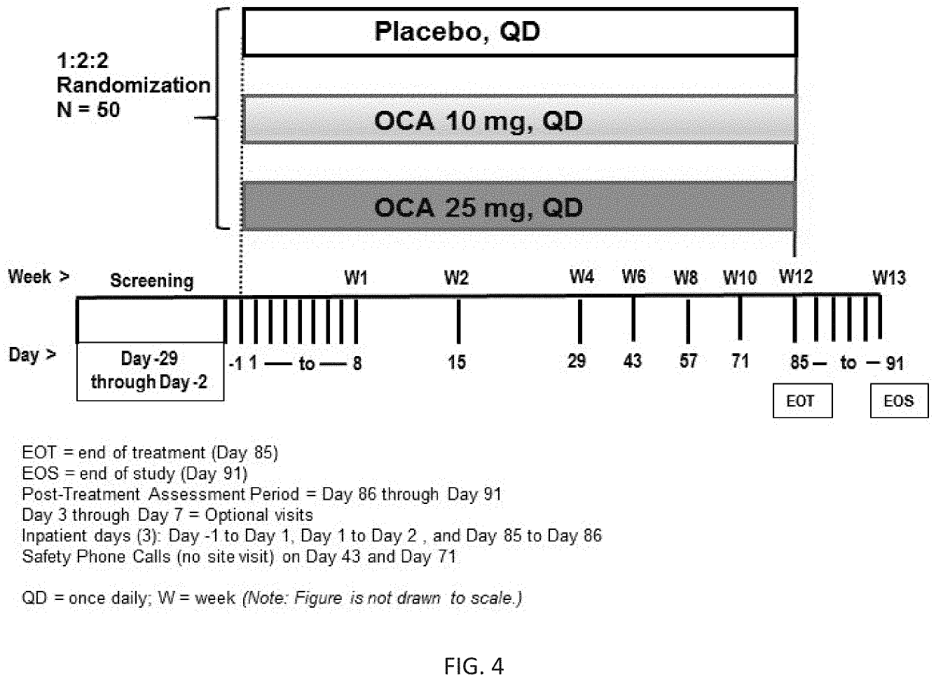

The study comprised three treatment groups-10 mg QD, 25 mg QD and Placebo QD. An outline of the study, including a comprehensive timeline is shown in FIG. 4.

The key inclusion and key exclusion criteria are shown in Table 1.

TABLE 1
Key Inclusion and Key Exclusion Criteria

| | |
|---|---|
| Key Inclusion Criteria | Histologic evidence of NASH - |
| | central read of liver biopsy ≤2 year |
| | prior to randomization |
| | Histologic evidence of fibrosis stage |
| | 1 to stage 4 without evidence of |
| | hepatic decompensation (NASH |
| | CRN criteria) |
| | If Type 2 diabetes mellitus: stable |
| | dose of anti-diabetic medications |
| Key Exclusion Criteria | LDL ≥190 mg/dL and statin therapy |
| | at Screening and/or proprotein |
| | convertase subtilisin/kexintype 9 |
| | inhibitor for ≥30 days at Screening |
| | CP score ≥7 within first 12 weeks of |
| | study |
| | HbA1c >9.5% within 28 days before |
| | Day-1 |
| | Evidence other known chronic liver |
| | disease |
| | Hepatic decompensation |
| | AST or ALT ≥7x ULN |
| | Total bilirubin >2 mg/dL |
| | Albumin <3.2 g/dL |
| | Serum Creatinine ≥1.5 mg/dL or Cr |
| | clearance <60 mL/min |
| | Platelet count <100,000/mm3 |
| | Ileal resection, or bariatric surgery |
| | BMI >45 kg/m2 and BP ≥140/90 |
| | mmHg if <60 years, ≥150/90 mmHg |
| | if ≥60 years, or on antihypertensive; |
| | LDL ≥160 mg/dL, T. chol. ≥200 |
| | mg/dL; or on lipid lowering; T2DM |

51 subjects were randomized into 1:2:2 placebo, OCA 10 mg QD or OCA 25 mg QD for 85 days as shown in Table 2 and FIG. 4. 17%, 29% and 5% of the subjects had a fibrosis score of F1, F2/F3 and F4 (mild hepatic impairment based on Child-Pugh Score [CP-A]), respectively.

TABLE 2
Demographics of three treatment groups

| | Placebo | 10 mg OCA | 25 mg OCA |
|---|---|---|---|
| N | 11 | 20 | 20 |
| Age | 53.5 (12.6) | 54.8 (13.6) | 54.8 (9.3) |
| Sex (M:F) | 3:8 | 2:18 | 8:12 |
| BMI | 34.6 (6.01) | 36.0 (4.4) | 34.5 (5.0) |
| Race | 100% White | 90% White | 95% White |
| NAS Score | 5.5 (0.71) | 5.0 (1.1) | 4.7 (1.4) |
| F1:F2:F3:F4 | 4:1:5:1 | 5:5:7:3 | 8:3:8:1 |
| Total Bilirubin (≤ULN) | Normal | Normal | Normal |
| Direct Bilirubin (≤ULN) | Normal | Normal | Normal |

Table 3 shows the TEAE incidence in the three different treatment groups.

TABLE 3
TEAE incidence rates in the three different treatment groups

| | Placebo | OCA 10 mg | OCA 25 mg |
|---|---|---|---|
| | (n = 11) | (n = 20) | (n = 20) |
| | n (%) | n (%) | n (%) |
| Total Number of TEAEs | 36 | 29 | 36 |
| Subjects with at least | 7 (64%) | 12 (60%) | 15 (75%) |
| one TEAE | | | |
| Pruritis | 4 (36%) | 3 (15%) | 5 (25%) |
| Nausea | 4 (36%) | 0 | 0 |
| Headache | 3 (27%) | 0 | 0 |
| Fatigue | 2 (18%) | 2 (10%) | 3 (15%) |
| Constipation | 1 (9%) | 2 (10%) | 2 (10%) |
| Low density lipoprotein | 0 | 2 (10%) | 2 (10%) |
| increased | | | |
| Pneumonia | 0 | 0 | 2 (10%) |

Table 4 shows a summary of the TEAEs of interest by SOC, PT and Maximum Severity Safety Population

TABLE 4
Summary of TEAEs of interest

| | Placebo | OCA 10 mg | OCA 25 mg |
|---|---|---|---|
| | (n = 11) | (n = 20) | (n = 20) |
| | n (%) | n (%) | n (%) |
| Any Serious TEAE | 0 | 0 | 0 |
| Subjects with at least one | 7 (64%) | 12 (60%) | 15 (75%) |
| TEAE | | | |
| Fatigue | 2 (18%) | 2 (10%) | 3 (15%) |
| Pruritus | | | |
| Mild | 3 (27%) | 3 (15%) | 2 (10%) |
| Moderate | 1 (9%) | 0 | 3 (15%) |
| Severe | 0 | 0 | 0 |
| Diabetes mellitus | 0 | 1 (5%) | 0 |
| Hepatobiliary disorders | 0 | 0 | 0 |
| (SOC) | | | |
| Constipation | 1 (9%) | 2 (10%) | 2 (10%) |
| Hypercholesterolemia | 0 | 0 | 1 (5%) |
| LDL Increased | | | |
| Mild | 0 | 2 (10%) | 1 (5%) |
| Moderate | 0 | 0 | 1 (5%) |
| Severe | 0 | 0 | 0 |

The results shown in Tables 3 and 4 indicate that OCA administered at 10 mg QD and 25 mg QD for 12 weeks is safe and well tolerated with no serious TEAE, no TEAEs within hepatic disorders and no TEAEs leading to study discontinuation. Furthermore, the number of TEAE's were similar across treatment groups and the incidence of pruritus, constipation and fatigue were similar across treatment groups. There was an increase in LDL cholesterol observed in the OCA treatment groups.

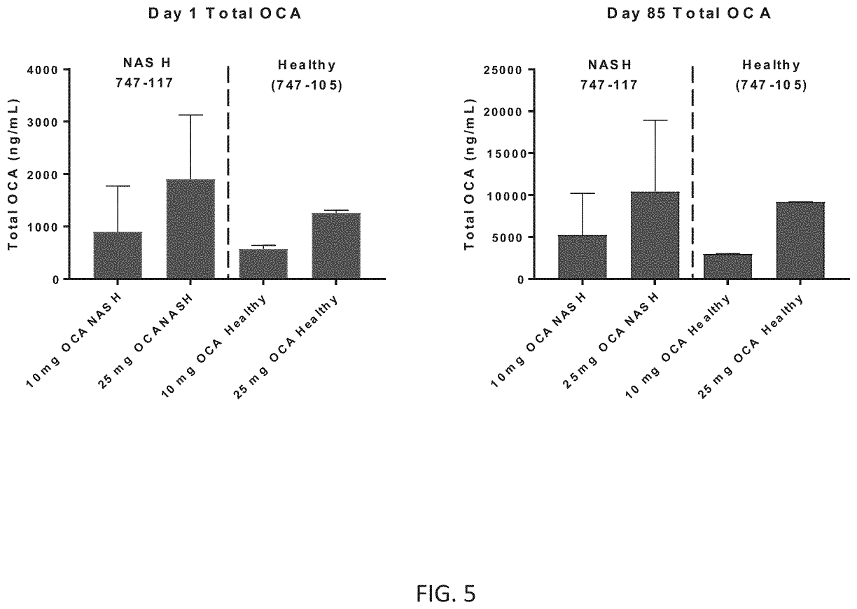

FIG. 5 shows the total OCA (ng/ml) in samples from healthy and NASH subjects administered OCA at a dose of 10 mg QD or 25 mg QD on day 1 of the present study and at the conclusion (day 85) of the present study. The results shown in FIG. 5 demonstrate that although there was some variability, plasma exposure of OCA was modestly higher in NASH subjects and accumulation was approximately 5-fold, which is consistent with a 4-day half-life observed in healthy subjects.

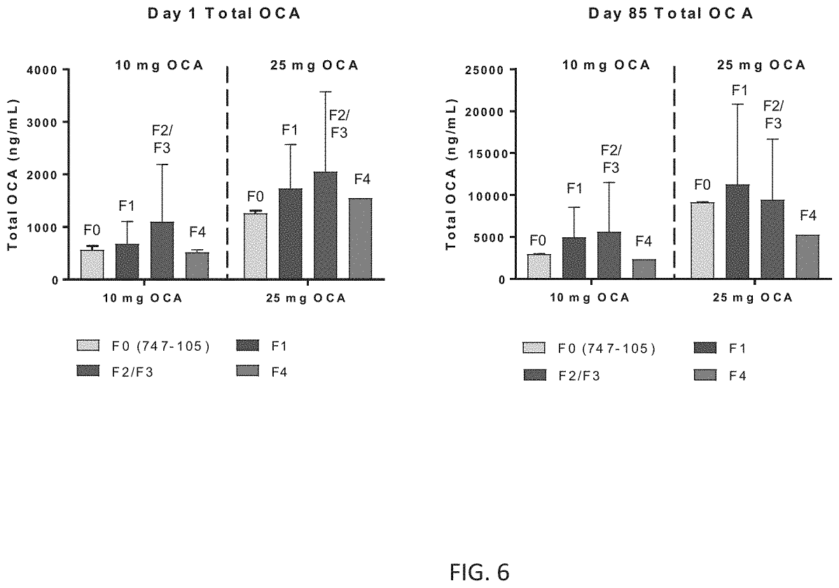

FIG. 6 shows the total OCA (ng/ml) in samples from NASH subjects with fibrosis scores of either F1, F2/F3 or F4 administered OCA at a dose of 10 mg QD or 25 mg QD on day 1 of the present study and at the conclusion (day 85) of the present study. The results shown in FIG. 6 demonstrate that although there was some variability, OCA exposure was generally highest in subjects with a F2/F3 fibrosis score. Furthermore, although there were a limited number of subjects having a fibrosis score of F4, plasma exposure was lower in these subjects as compared to the subjects with a fibrosis score of F1 or F2/F3.

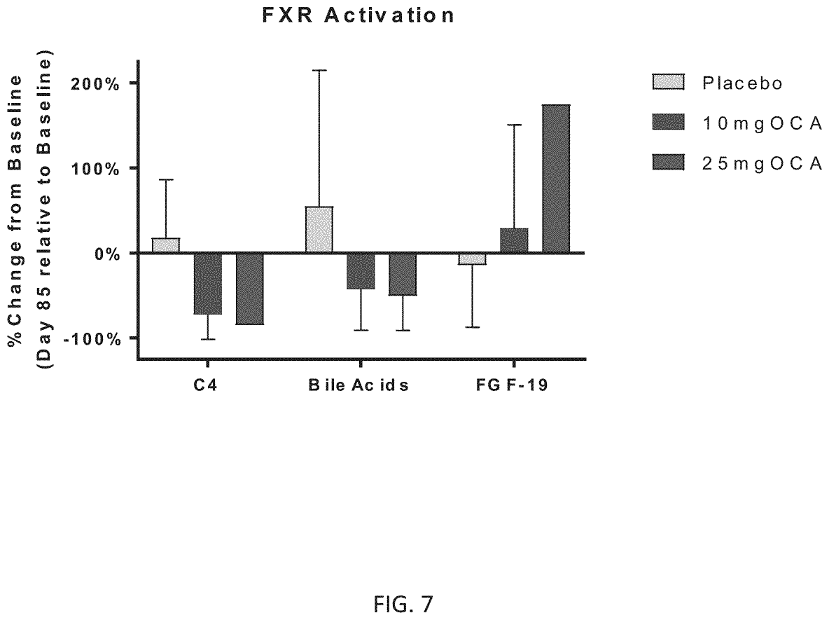

FIG. 7 shows the percent change from baseline (Day 85 compared to baseline) in C4 levels, bile acid levels and FGF-19 levels in the three treatment groups of the present study. The results shown in FIG. 7 demonstrate that although OCA suppressed C4 levels (a marker of bile acid synthesis and liver FXR activation), no dose response was observed. Furthermore, plasma bile acids were suppressed by administration of OCA, but no dose response was observed. Finally, FGF-19, a marker of FXR activation in the intestine, increased with OCA dose.

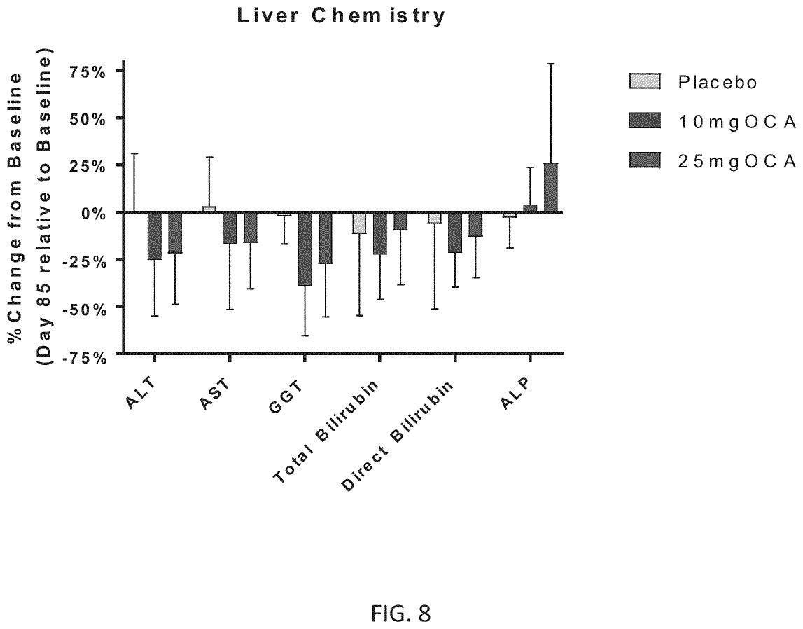

FIG. 8 shows the percent change from baseline (Day 85 compared to baseline) in ALT, AST, GGT, total bilirubin, direct bilirubin and ALP levels in the three treatment groups of the present study. The results shown in FIG. 8 demonstrate that although OCA suppressed biochemistry markers of hepatic cytotoxicity and dysfunction, no dose-dependent response was observed. However, a dose dependent increase in ALP was observed upon administration of OCA.

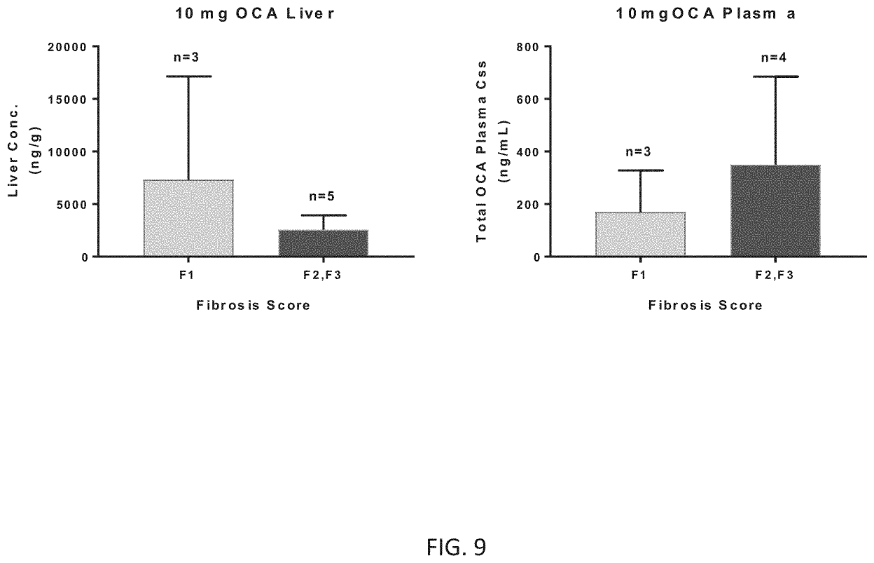

FIG. 9 shows the liver exposure of OCA and its conjugates in liver and plasma samples from NASH subjects with fibrosis scores of F1 or F2/F3 that were administered 10 mg OCA QD. The results shown in FIG. 9 demonstrate that the liver exposure of OCA was generally similar across subjects with different fibrosis scores at the 10 mg QD dose.

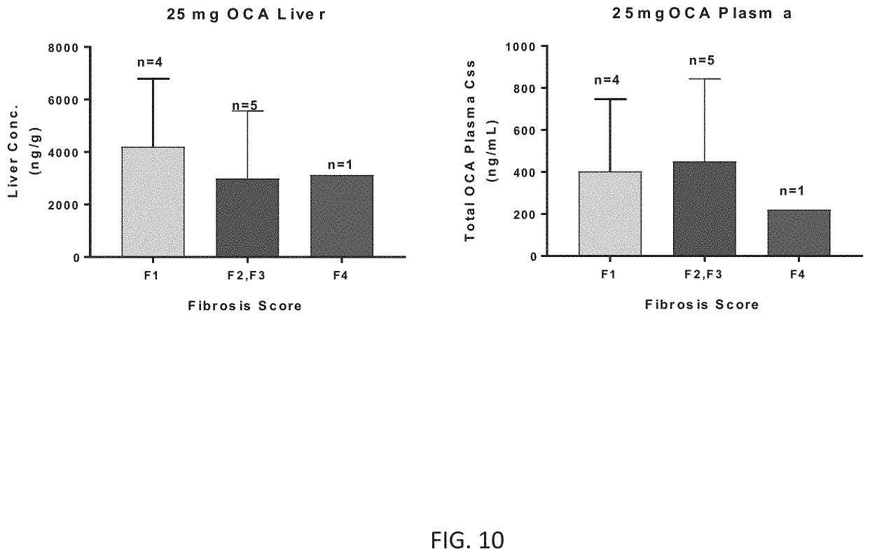

FIG. 10 shows the liver exposure of OCA and its conjugates in liver and plasma samples from NASH subjects with fibrosis scores of F1, F2/F3 or F4 that were administered 25 mg OCA QD. The results shown in FIG. 10 demonstrate that the liver exposure of OCA was generally similar across subjects with different fibrosis scores at the 25 mg QD dose.

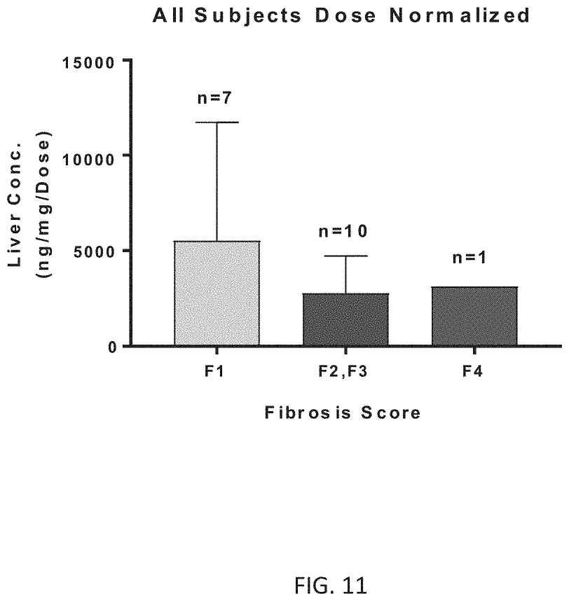

FIG. 11 shows the liver exposure of OCA and its conjugates in liver samples of NASH subjects with fibrosis scores of F1, F2/F3 or F4 after normalization by dose. The results shown in FIG. 11 demonstrate that liver exposure of OCA was generally similar across subjects with different fibrosis scores after correcting for dose.

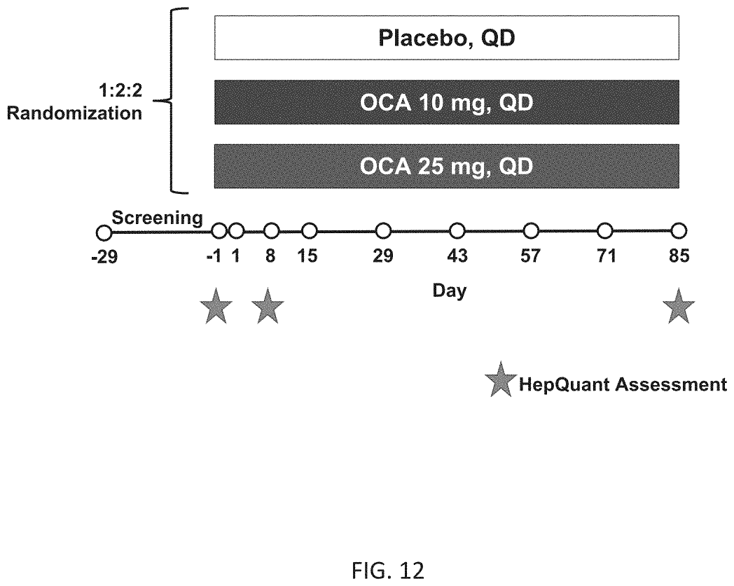

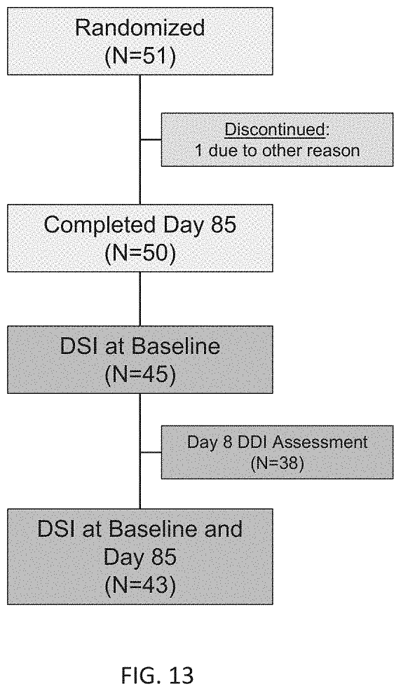

As shown in FIG. 12, subjects enrolled the study were also administered HepQuant®-SHUNT tests one day prior to the initiation of treatment (−1), eight days after initiation of treatment (8) and at the end of treatment (85). As part of the HepQuant®-SHUNT test, labeled cholate was administered intravenously and orally. The HepQuant®-SHUNT tests were used to determine disease severity index (DSI) scores. As shown in FIG. 13, 43 of the 51 subjects enrolled in the study were administered the HepQuant®-SHUNT tests. The demographics of these 43 subjects are shown in Table 5

TABLE 5
Demographics of subjects administered HepQuant ®-SHUNT tests.

| | Placebo | | OCA 10 mg | | OCA 25 mg | |
|---|---|---|---|---|---|---|
| | (n = 10) | | (n = 16) | | (n = 17) | |
| Age, years | 57 | (12) | 5 | (14) | 56 | (9) |
| Female, n (%) | 7 | (70) | 1 | (100) | 10 | (60) |
| White, n (%) | 10 | (100) | 14 | (88) | 17 | (100) |
| BMI, kg/m² | 34 | (5) | 36 | (4) | 34 | (4) |
| Diabetes at Baseline, n (%) | 5 | (50) | 14 | (88) | 10 | (59) |
| Liver Fibrosis Stage, n (%) | | | | | | |
| Stage 1 | 4 | (40) | 3 | (19) | 5 | (29) |
| Stage 2 | 0 | | 4 | (25) | 3 | (18) |
| Stage 3 | 5 | (50) | 7 | (44) | 8 | (47) |
| Stage 4 | 1 | (10) | 2 | (13) | 1 | (6) |
| NAS | 5 | (1) | 5 | (1) | 5 | (1) |
| ALT, U/L | 63 | (38) | 48 | (23) | 41 | (20) |
| AST, U/L | 53 | (30) | 41 | (19) | 32 | (15) |
| ALP, U/L | 115 | (35) | 90 | (30) | 93 | (34) |
| Total Bilirubin, μmol/L | 13 | (7) | 9 | (4) | 9 | (3) |
| INR | 1 | (0.1) | 1 | (0.1) | 1 | (0.1) |
| Platelets, ×10⁹/L | 201 | (73) | 215 | (62) | 219 | (56) |

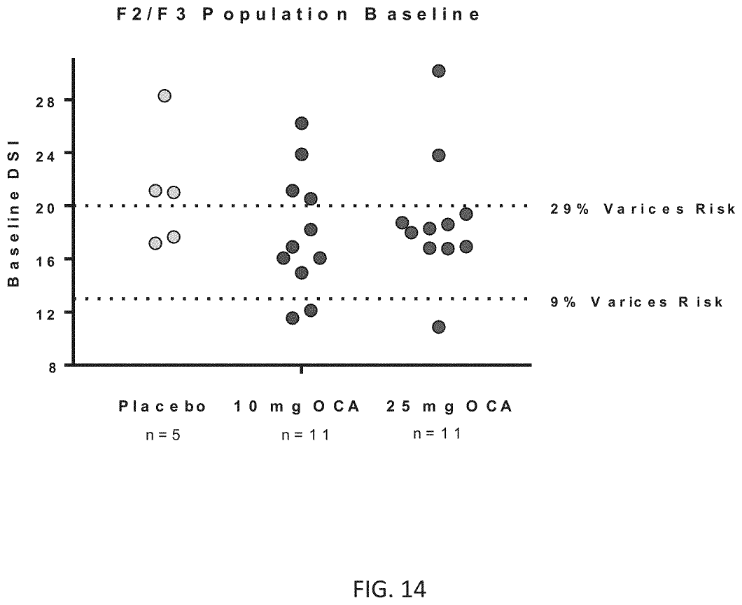

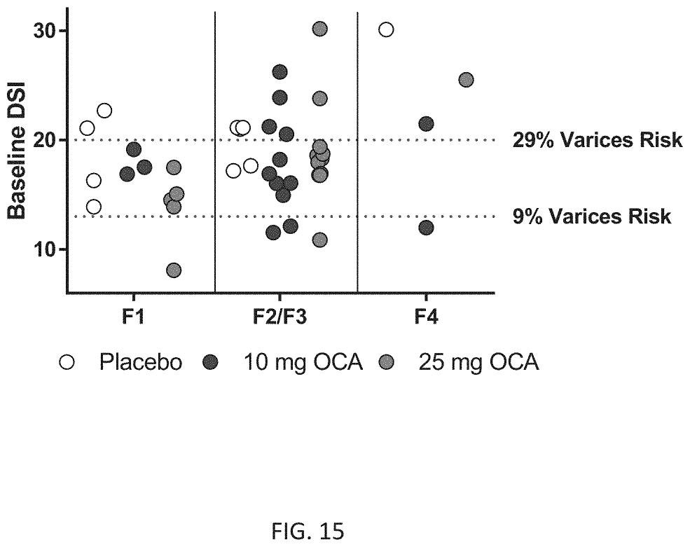

FIG. 14 shows the baseline DSI score in NASH subjects having a fibrosis score of F2/F3 in the three treatment groups of the present study. The results shown in FIG. 14 show that the F2/F3 NASH subjects had a higher baseline risk of varices and the risk was consistent across the different treatment groups. FIG. 15 shows the Baseline DSI score in NASH subjects having a fibrosis scores of F1, F2/F3 or F4 in the three treatment groups of the present study. The results shown in FIG. 15 are consistent with a wide range of hepatic dysfunction across each stage of fibrosis.

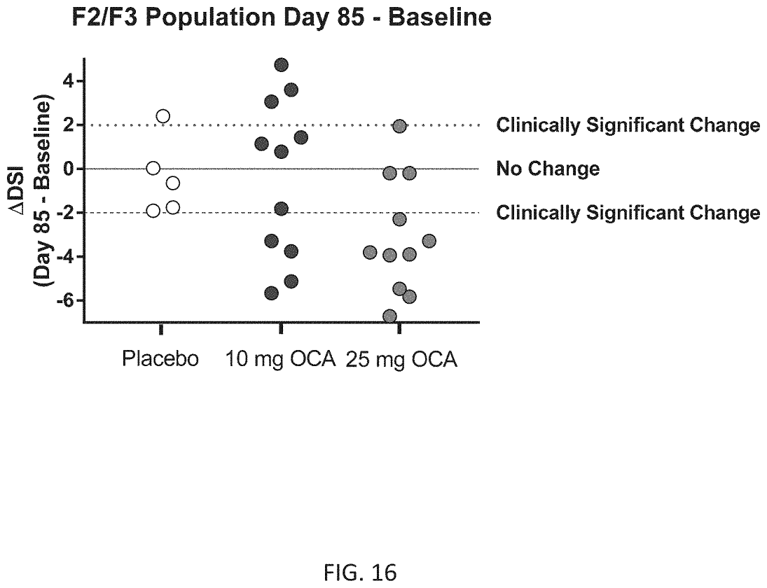

FIG. 16 shows the change in DSI score (Day 85−Baseline [Day 1]) in NASH subjects having a fibrosis score of F2/F3 in the three treatment group of the present study. The results in FIG. 16 show that there was a greater improvement in DSI score for subject treated with OCA, with the greatest effect seen in the subjects administered 25 mg QD.

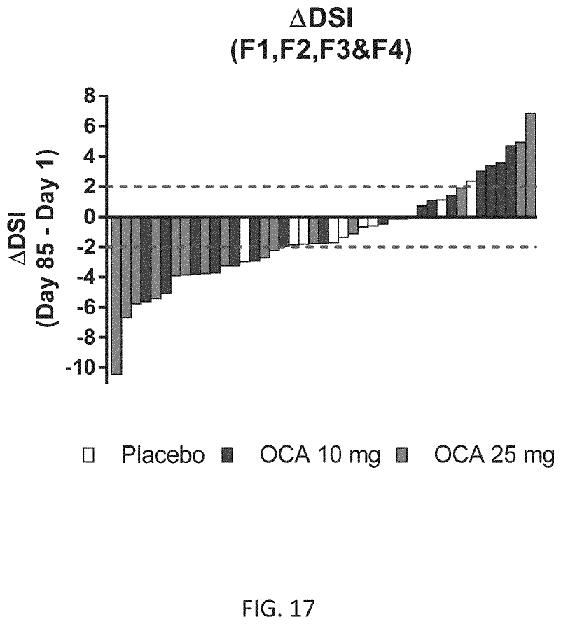

FIG. 17 shows the change in DSI score (ΔDSI, Day 85−Baseline [Day 1]) in NASH subjects having a fibrosis score of F1, F2/F3 or F4 in the three treatment groups of the present study. In total, 18 of the subjects showed a clinically relevant ΔDSI of at least −2. Of these 18 subjects, 17 of them were treated with OCA, with subjects treated with 25 mg QD showing a greater ΔDSI than subjects treated with 10 mg QD. Table 6 shows the responders sorted by fibrosis stage. These results indicate that a dose-dependent and clinically meaningful reduction in DSI was observed in subjects administered 10 mg QD or 25 mg QD, with a greater response shown in subjects administered 25 mg QD.

TABLE 6
OCA Responders

| Patient Groups | Placebo | OCA 10 mg | | OCA 25 mg | |
|---|---|---|---|---|---|
| All | n = 10 | n = 16 | | n = 17 | |
| Responders, n (%) | 1 (10) | 7 | (44) | 10 | (59) |
| F1 | n = 4 | n = 3 | | n = 5 | |
| Responders, n (%) | 1 (25) | 2 | (67) | 1 | (20) |
| F2/F3 | n = 5 | n = 11 | | n = 11 | |
| Responders, n (%) | 0 | 4 | (36) | 8 | (73) |
| F4 | n = 1 | n = 2 | | n = 1 | |
| Responders, n (%) | 0 | 1 | (50) | 1 | (100) |

In summary, 50 subjects, primarily white with median age of 55 and BMI 35.1 kg/m2 completed Day 85 of the present study. Of those subjects, 38 at Day 8 and 43 at Day 85 had HepQuant assessments. The mean baseline DSI±SD (n) was for F1 16.4±3.8 (n=12), F2/3 19.0±4.6 (n=28), and F4 22.1±6.7 (n=5). The mean baseline DSI score for all subjects (F1-F4) was consistent with a probability of approximately 20 to 40% for any varices (small or large) based on previous results in NASH and HCV subjects. At Day 85 a dose-dependent response was observed for F2/F3 subjects obtaining a ≥2 point decrease in DSI (4 [36%] OCA 10 mg, 8 [73%] OCA 25 mg vs 0 (0%) placebo). A similar trend was observed in F1-F4 subjects (7 [44%] OCA 10 mg, 10 [59%] OCA 25 mg vs 1 [10%] placebo). Thus, OCA was found to be generally safe and well tolerated. A fibrosis dependent increase in the baseline DSI was observed. A dose-dependent response was observed for subjects obtaining a ≥2 point decrease in DSI on Day 85.

1. A method of treating a disease in a subject comprising:
a) determining a Disease Severity Index (DSI) score using a HepQuant test;
b) comparing the DSI score to a predetermined cutoff value; and
c) administering to the subject at least one therapeutically effective amount of obeticholic acid, or a pharmaceutically acceptable salt, ester, or amino acid conjugate thereof when the DSI score is equal to or greater than the predetermined cutoff value.

2. A method of treating a disease in a subject comprising:
a) determining a first Disease Severity Index (DSI) score using a HepQuant test at a time point prior to the administration of at least one therapeutically effective amount of obeticholic acid, or a pharmaceutically acceptable salt, ester, or amino acid conjugate thereof;
b) administering to the subject at least one therapeutically effective amount of obeticholic acid, or a pharmaceutically acceptable salt, ester, or amino acid conjugate thereof;
c) determining at least a second DSI score using a HepQuant test at least one time point after the administration of the at least one therapeutically effective amount of obeticholic acid, or a pharmaceutically acceptable salt, ester, or amino acid conjugate thereof;
d) determining a DSI difference score by calculating the difference between the at least second DSI score and the first DSI score;
e) comparing the DSI difference score to a predetermined cutoff value;
f) discontinuing administration of the at least one therapeutically effective amount of obeticholic acid when the DSI difference score is less than or equal to the predetermined cutoff value, or continuing administration of the at least one therapeutically effective amount of obeticholic acid when the DSI difference score is greater than or equal to the predetermined cutoff value.

3. (canceled)

4. A method of treating a disease in a subject comprising:
a) determining a first Disease Severity Index (DSI) score using a HepQuant test at a time point prior to the administration of at least one first therapeutically effective amount of obeticholic acid, or a pharmaceutically acceptable salt, ester, or amino acid conjugate thereof;
b) administering to the subject at least one first therapeutically effective amount of obeticholic acid, or a pharmaceutically acceptable salt, ester, or amino acid conjugate thereof;
c) determining at least a second DSI score using a HepQuant test at least one time point after the administration of the at least one first therapeutically effective amount of obeticholic acid, or a pharmaceutically acceptable salt, ester, or amino acid conjugate thereof;
d) determining a DSI difference score by calculating the difference between the at least second DSI score and the first DSI score;
e) comparing the DSI difference score to a predetermined cutoff value;
f) administering to the subject at least one second therapeutically effective amount of obeticholic acid, or a pharmaceutically acceptable salt, ester, or amino acid conjugate thereof when the DSI difference score is greater than or equal to the predetermined cutoff value, or administering to the subject at least one second therapeutically effective amount of obeticholic acid, or a pharmaceutically acceptable salt, ester, or amino acid conjugate thereof when the DSI difference score is less than or equal to the predetermined cutoff value.

5. (canceled)

6. The method of claim 1, wherein the disease is a liver disease.

7. The method of claim 6, wherein the liver disease is chronic liver disease, nonalcoholic fatty liver disease (NAFLD), nonalcoholic steatohepatitis (NASH), hepatitis C infection, alcoholic liver disease, liver damage due to progressive fibrosis, or liver fibrosis.

8. The method of claim 7, wherein the liver disease is NASH.

9. The method of claim 8, wherein the NASH is NASH with mild fibrosis, NASH with moderate/severe fibrosis or NASH with cirrhosis.

10. The method of claim 8, wherein the NASH has a fibrosis score of F1, F2, F3, F2/F3 or F4.

11. The method of claim 1, wherein the HepQuant test is a HepQuant-SHUNT test or a HepQuant-STAT test.

12. The method of claim 11, wherein the HepQuant test is a HepQuant-SHUNT test.

13. The method of claim 1, wherein HepQuant test comprises administering to the subject labeled cholate.

14. The method of claim 13, wherein the HepQuant test comprises administering d4-cholate and 13C-cholate.

15. The method of claim 14, wherein the d4-cholate is administered orally and the 13C-cholate is administered intravenously.

16. The method of claim 1, wherein the therapeutically effective amount of obeticholic acid is 10 mg once daily.

17. The method of claim 1, wherein the therapeutically effective amount of obeticholic acid is 25 mg once daily.

18. The method of claim 4, wherein the first therapeutically effective amount is less than the second therapeutically effective amount.

19. The method of claim 4, wherein the first therapeutically effective amount is greater than the second therapeutically effective amount.

20. The method of claim 4, wherein the first therapeutically effective amount is the same as the second therapeutically effective amount.
